(19) United States
(12) Patent Application Publication
(10) Pub. No.: US 2005/0256112 A1
(43) Pub. Date: Nov. 17, 2005

(54) Combination of atypical antipsychotics and 5HT-1B receptor antagonists

(75) Inventors: Michael A. Brodney, East Lyme, CT (US); Christopher J. Helal, East Lyme, CT (US); Brian S. Bronk, Gales Ferry, CT (US); Spiros Liras, Stonington, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/128,146

(22) Filed: May 11, 2005

Related U.S. Application Data
(60) Provisional application No. 60/569,927, filed on May 11, 2004.

(51) Int. Cl. A61K 31/541

(52) U.S. Cl. 514/227.5

(57) ABSTRACT

The present invention relates to a pharmaceutical composition for treating, for example, a disorder or condition selected from the group consisting of hypertension, depression, generalized anxiety disorder, phobias, posttraumatic stress disorder, avoidant personality disorder, sexual dysfunction, eating disorders, obesity, chemical dependencies, cluster headache, migraine, pain, Alzheimer's disease, obsessive-compulsive disorder, panic disorder, memory disorders, Parkinson's diseases, endocrine disorders, cerebellar ataxia, gastrointestinal tract disorders, negative symptoms of schizophrenia, premenstrual syndrome, Fibromyalgia Syndrome, stress incontinence, Tourette syndrome, trichotillomania, kleptomania, male impotence, cancer, chronic paroxysmal hemicrania and headache in a mammal, preferably a human, comprising (i) an atypical antipsychotic or a pharmaceutically acceptable salt thereof,
(ii) a 5-HT_{1B} receptor antagonist or a pharmaceutically acceptable salt thereof, wherein the 5-HT_{1B} receptor antagonist is selected from the group consisting of (A) a compound of the formula I as described in the specification and (B) a compound of the formula II as described in the specification, and optionally (iii) a pharmaceutically acceptable carrier.

This application claims priority under 35 U.S.C 119 of U.S. Provisional 60/569,927 filed May 11, 2004. The entire contents of the prior application are incorporated herein by reference.

### FIELD OF THE INVENTION

The present invention relates to pharmaceutical compositions containing an atypical antipsychotic or pharmaceutically acceptable salts thereof and 5-HT_{1B} receptor antagonists or pharmaceutically acceptable salts thereof, and to their medicinal use for treating disorders associated with the central nervous system.

### BACKGROUND OF THE INVENTION

I. 5-HT_{1B} Receptor Antagonists:

U.S. Pat. Nos. 6,464,028, 6,258,953, 6,380,186, 6,323,229, 6,197,773, 6,451,803, 6,403,592, 6,472,388, 6,562,813 and 6,627,627 and U.S. Patent Publication Nos. 2002/0091119 and 2003/0083337 describe certain aralkyl and aralkylidene heterocyclic lactams and imides that are 5-HT_{1B} receptor antagonists and that are used in the compositions of the present invention. Other 5-HT, receptor antagonists are described in European Patent Publications 701,819, 434,561 and 343,050, PCT publications WO 94/21619, WO 95/31988, and WO 96/00720, Glennon et al., “5-HT_{1D} Serotonin Receptors”, Clinical Drug Res. Dev., 22, 25-36 (1991), and G Maura et al., J. Neurochem, 66 (1), 203-209 (1996). These references describe 5-HT, receptor antagonists, including 5-HT_{1B} receptor antagonists, as useful in the treatment of, for example, migraine, depression, obsessive compulsive disorder, post-traumatic stress disorder (PTSD), and eating disorders, as well as other disorders associated with the central nervous system.

II. Atypical Antipsychotic Medications:

Typical antipsychotic compounds are well-known in the art and include drugs derived from phenothiazines, such as thioridazine and perphenazine; butyrophenone-derived compounds, such as haloperidol (Haldol); and compounds of the diphenylbutylpiperdine group, such as pimozide. The compounds are dopamine antagonists, binding to dopamine (D2) receptors, thereby blocking the receptors and reducing or preventing receptor-dopamine binding. When used in the treatment of psychotic disorders, the compounds function to very effectively reduce “positive symptoms” of schizophrenia and related psychotic disorders, including delusions and hallucinations.

The side effects caused by the typical antipsychotics are considerable, and can be life-threatening. Patients may suffer from akathisia, dystonias, muscle rigidity and shuffling gait, some of which is irreversible. Significant weight gain is a side effect also associated with the use of typical antipsychotics. The frequent occurrence of uncomfortable or unmanageable side effects often results in reduced compliance with, or increased cost of, the drug treatment regime.

Recently, new compounds for use in the treatment of psychotic disorders have been developed. These compounds, designated “atypical” antipsychotics, to distinguish them from the “typical” or older antipsychotic medications, are primarily benzisoxals, and are characterized by their antagonistic action on multiple receptors, including the serotonin (5HT2) receptors and the dopamine (D2) receptors of the central nervous system. Some of the compounds, including risperidone, also act as blockers of the central andrengenic receptors. The current list of atypical antipsychotic drugs is well known in the art and includes, but is not limited to, azenapine, clozapine (Clozaril®), olanzapine (Zyprexa®) quetiapine (Seroquel®) and ziprasidone (Geddon®). The precise chemical compositions and configurations of these compounds can be found in the Merck Index, 12^{th} ed., 1996, and are incorporated herein by reference.

An additional atypical antipsychotic, also well known in the art, is risperidone, sold under the trade name “Risperdal®)” by Janssen Pharmaceuticals of Beerse, Belgium. Classified as a benzisoxazol and an atypical antipsychotic, risperidone has the properties to not only block D2 receptors, but 5HT2 receptors as well. This medication is extensively metabolized in the liver by the cytochrome P450IID6 to the principle metabolite, 9-hydroxyrisperidone. Further chemical properties and the structure of risperidone are discussed in U.S. Pat. No. 4,804,663 to Kennis et al., issued Feb. 14, 1989, entitled “3-piperidinyl-substituted 1,2,-benzisoxazoles and 1,2-benzisothiazoles,” the contents of which are incorporated herein by reference. The chemical designation of risperidone is 3-[2-[4-(6-fluoro-1,2-benzisoxazol-3-yl)-1-piperidinyl]ethyl]-6,7,8,9-tetrahydro-2-methyl-4H-pyrido[1,2-a]pyrimidin-4-one.

Like their “typical” counterparts, the atypical antipsychotics have been shown to reduce the occurrence of “positive” side effects in individuals suffering from psychotic disorders. They also have been shown to reduce the “negative” symptoms of schizophrenia, including social isolation, emotional withdrawal, decreased motivation, and subnormal communication and social skills.

With some exceptions, the side effect profiles of the atypical antipsychotics are highly favorable compared to those of the typical antipsychotics. However, clozapine reduces white blood cell counts, so its administration must be accompanied by costly blood tests to monitor for potentially fatal agranulocytosis. Olanzapine has been shown to cause significant weight gain, in some cases up to 1 pound per week and is, therefore, not particularly suitable for use in a population of patients specifically fearing weight gain. Quetiapine has been shown to cause cataract formation in some mammals. In contrast, risperidone has been shown to have few of these side effects. White blood cell count remains unaffected and weight gain is minimal. The few side effects attributable to risperidone can be easily monitored and corrected.

There is a present need to develop new methods of treating CNS disorders. The present invention achieves this goal.

### SUMMARY OF THE INVENTION

The present invention relates to a pharmaceutical composition for treating a CNS disorder, comprising:
(i) an atypical antipsychotic or a pharmaceutically acceptable salt thereof,
(ii) a 5-HT_{1B} receptor antagonist or a pharmaceutically acceptable salt thereof, wherein the 5-HT_{1B} receptor antagonist is selected from the group consisting of
(A) a compound of the formula I— 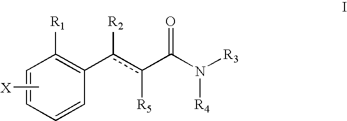
   wherein, in formula I:
R¹ is a group of the formula G¹, G², G³, G⁴, G⁵, G ⁶ or G⁷ depicted below, 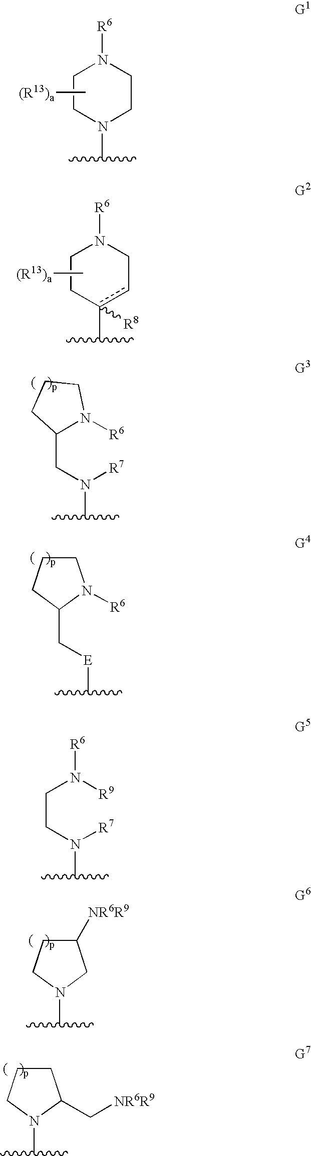
   a is zero to eight;
each R¹³ is, independently, (C₁ -C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G² respectively, having an available bonding site, or to a ring carbon of R⁶ having an available bonding site;

E is oxygen, sulfur, SO or SO₂;
X is hydrogen, chloro, fluoro, bromo, iodo, cyano, (C₁-C₆)alkyl, hydroxy, trifluoromethyl, (C₁-C₆)alkoxy, —SOₜ(C₁-C₆)alkyl wherein t is zero, one or two, —CO₂R¹⁰ or —CONR¹¹R¹²,
R² is hydrogen, (C₁-C₄)alkyl, phenyl or naphthyl, wherein said phenyl or naphthyl is optionally substituted with one or more substituents independently selected from the group consisting of chloro, fluoro, bromo, iodo, (C₁-C₆)alkyl, (C₁-C₆)alkoxy, trifluoromethyl, cyano and —SOₖ(C₁-C₆)alkyl wherein k is zero, one or two;
R³ is —(CH₂)ₘB, wherein m is zero, one, two or three and B is hydrogen, phenyl, naphthyl or a 5 or 6 membered heteroaryl group containing from one to four hetero-atoms in the ring, and wherein each of the foregoing phenyl, naphthyl and heteroaryl groups is optionally substituted with one or more substituents independently selected from the group consisting of chloro, fluoro, bromo, iodo, (C₁-C₆)alkyl, (C₁-C₆)alkoxy, (C₁-C₆) alkoxy-(C₁-C₆)alkyl-, trifluoromethyl, trifluoromethoxy, cyano, hydroxy, —COOH and —SOₙ(C₁-C₆)alkyl wherein n is zero, one or two;
R⁴is (C₁-C₆)alkyl or C₆-C₁₀ aryl;
or R³ and R⁴ can optionally be taken together with the nitrogen to which they are attached to form a five to seven membered heteroalkyl ring, wherein any two of the carbon atoms of said heteroalkyl ring is optionally replaced with a heteroatom selected from the group consisting of nitrogen, oxygen or sulfur (e.g., pyrrolidine, isoxazolidine, 1,3-oxazolidin-3-yl, isothiazolidine, 1,3-thiazolidin-3-yl, 1,2-pyrazolidin-2-yl, 1,3-pyrazolidin-1-yl, piperidine, thiomorpholine, 1,2-tetrahydrothiazin-2-yl, 1,3-tetrahydrothiazin-3-yl, tetrahydrothiadiazine, morpholine, 1,2-tetrahydrodiazin-2-yl, 1,3-tetrahydrodiazin-1-yl, piperazine, etc.); wherein said heteroalkyl ring can optionally be substituted by aryl or heteroaryl (e.g., furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, benzoxazolyl, benzothiazolyl, benzisothiazolyl, benzisoxazolyl, indanyl, benzimidazolyl, thianaphthenyl, isothianaphthenyl, benzofuranyl, isobenzofuranyl, isoindolyl, indolyl, indazolyl, isoquinolyl, quinolyl, phthalazinyl, quinoxalinyl, quinazolinyl, benzoxazinyl; etc.);
R⁵ is hydrogen, (C₁-C₆)alkyl or aryl, wherein aryl is selected from the group consisting of phenyl, naphthyl, pyridyl or pyrimidyl, wherein any of said aryl is optionally independently substituted on any available bonding site by any of the radicals of X;
or R⁵ and R⁴ taken together form a divalent group —Yₙ₂—;
Y is selected from the group consisting of (a) CR⁴R⁵, wherein R⁴ and R⁵ are independently selected from hydrogen, (C₁-C₆)alkyl and trifluoromethyl; (b) a phenylene, naphthylene or a 5 or 6 membered heteroarylene ring comprising containing from one to four hetero-atoms in the heteroarylene ring, and wherein each of the foregoing phenylene, naphthylene and heteroarylene rings can optionally be substituted with one or more substituents independently selected from the group consisting of chloro, fluoro, bromo, iodo, (C₁-C₆)alkyl, (C₁-C₆)alkoxy, (C₁-C₆) alkoxy-(C₁-C₆)alkyl-, trifluoromethyl, trifluoromethoxy, cyano, hydroxy, —COOH and —SOₙ(C₁-C₆)alkyl wherein n is zero, one or two, wherein two adjacent ring atoms of ring Y are also ring atoms of ring A; and (c) an optionally substituted (C₁-C₄) heteroalkyl bridge that, together with the atoms to which it is attached, forms a five to seven membered heterocycle containing two to four heteroatoms selected from the group consisting of 1,3-oxazolidin-4-on-5-yl, 1,3-oxazolidin-2,4-dion-5-yl, 4,5-dihydro-1,2-oxazolidin-3-on-4-yl, 1,3-thiazolidin-4-on-5-yl, 1,3-thiazolidin-2,4-dion-5-yl, 1,3-pyrazolidin-4-on-5-yl, 1,3-imidazolidin-2,4-dion-5-yl, 1,2-pyrazolidin-3-on-4-yl, 1,2-thiazolidin-1,1,3-trion-4-yl, 1,2-thiazolidin-3-on-4-yl, tetrahydro-1,2-oxazin-3-on4-yl, tetrahydro-1,3-oxazin-4-on-5-yl, tetrahydro-1,3-oxazin-2,4-dion-5-yl, morpholin-3-on-2-yl, morpholin-3,5-dion-2-yl, 2,3-dihydro-1,4-oxazin-3-on-2-yl, tetrahydro-1,3-thiazin-4-on-5-yl, tetrahydro-1,3-thiazin-2,4-dion-5-yl, tetrahydro-1 ,2-thiazin-3-on-4-yl, thiomorpholin-3-on-2-yl, thiomorpholin-3, 5-dion-2-yl, 2,3-dihydro-1,4-thiazin-3-on-2-yl, hexahydro-1,2-diazin-3-on4-yl, 4,5-dihydro-2H-pyridazin-3-on-4-yl, hexahydro-1,3-diazin-4-on-5-yl, hexahydro-1,3-diazin-2,4-dion-5-yl, piperazin-2-on-3-yl, piperazin-2,6-dion-3-yl, tetrahydro-1,3,4-thiadiazin-5-on-6-yl, 5,6-dihydro-1,3,4-thiadiazin-5-on-6-yl, 1,3,4-oxadiazin-5-on-6-yl, 5,6-dihydro-1,2,4-oxadiazin-5-on-6-yl, tetrahydro-1,2,4-oxadiazin-5-on-6-yl, 1,2,4-triazin-5-on-6-yl, tetrahydro-1,2,4-oxadiazin-5-on-6-yl, 5,6-dihydro-1-2,4-oxadiazin-5-on-6-yl, 1,2,4-oxadiazin-3,5-dion-6-yl, 1,2,4-trazin-6-on-5-yl, hexahydro-1,2-oxazepin-3-on-2-yl, hexahydro-1,3-oxazepin-4-on-5-yl, hexahydro-1,4-oxazepin-3-on-2-yl, hexahydro-1,4-oxazepin-3,5-dion-2-yl, hexahydro-1,4-oxazepin-3,5-dion-6-yl, 2,3,5,6-tetrahydro-1-4-oxazepin-5,7-dion-6-yl, hexahydro-1,4-oxazepin-5-on-6-yl, hexahydro-1,3-oxazepin-2,4-dion-5-yl, hexahydro-1,2-thiazepin-3-on-4-yl, hexahydro-1,4-thiazepin-3-on-2-yl, 2,3,4,5-tetrahydro-1,4-thiazepin-3-on-2-yl, hexahydro-1,4-thiazepin-3,5-dion-2-yl, hexahydro-1,4-thiazepin-3,5-dion-6-yl, 2,3,6,7-tetrahydro-1,4-thiazepin-5-on-6-yl, 6,7-dihydro-1,4-thiazepin-5-on-6-yl, hexahydro-1,3-thiazepin-2,4-dion-5-yl, hexahydro-1,2-diazepin-3-on-4-yl, hexahydro-1,3-diazepin-2,4-dion-5-yl, hexahydro-1,4-diazepin-2-on-3-yl, hexahydro-1,4-diazepin-5-on-6-yl, hexahydro-1,4-diazepin-5,7-dion-6-yl, hexahydro-1,3,5-thiadiazepin-3-on-7-yl, 4,5,6,7-tetrahydro-1-3,5-thiadiazepin-6-on-7-yl, and 2,3,5,6-tetrahydro-1,2,4-triazepin-3,5-dion-7-yl; wherein the substituents on any of the carbon atoms capable of supporting an additional bond, of said (C₁-C₄) heteroalkyl bridge, are chloro, fluoro, (C₁-C₆)alkyl, (C₁-C₆)alkoxy, trifluoromethyl or cyano; wherein the substituents on any of the nitrogen atoms capable of supporting an additional bond, of said (C₁-C₄) heteroalkyl bridge, are (C₁-C₆)alkyl or trifluoromethyl,
n2 is one, two, three or four, with the proviso that n2 is one when Y is not CR⁴R⁵;

R⁶ is selected from the group consisting of hydrogen, (C₁-C₆)alkyl optionally substituted with (C₁-C₆)alkoxy or one to three fluorine atoms, or [(C₁-C₄)alkyl]aryl wherein the aryl moiety is phenyl, naphthyl, or heteroaryl-(CH₂)_{q}—, wherein the heteroaryl moiety is selected from the group consisting of pyridyl, pyrimidyl, benzoxazolyl, benzothiazolyl, benzisoxazolyl and benzisothiazolyl and q is zero, one, two, three or four, and wherein said aryl and heteroaryl moieties can optionally be substituted with one or more substituents independently selected from the group consisting of chloro, fluoro, bromo, iodo, (C₁-C₆)alkyl, (C₁-C₆)alkoxy, trifluoromethyl, cyano and —SO_{g}(C₁-C₆)alkyl, wherein g is zero, one or two;
R⁷ is selected from the group consisting of hydrogen, (C₁-C₆)alkyl, [(C₁-C₄)alkyl]aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties can optionally be substituted with one or more substituents independently selected from the group consisting of chloro, fluoro, bromo, iodo, (C₁-C₆)alkyl, (C₁-C₆)alkoxy, trifluoromethyl, —C(═O)—(C₁-C₆)alkyl, cyano and —SOⱼ(C₁-C₆)alkyl, wherein j is zero, one or two;
or R⁶ and R⁷ taken together form a C₂-C₄ alkylene chain;
R⁸ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that contains, in addition to the nitrogen atom to which R⁶ and R⁹ are attached, from zero to four heteroatoms selected from the group consisting of nitrogen, sulfur and oxygen;
and p is one, two, or three;
each of R¹⁰, R¹¹ and R¹² is selected, independently, from the groups set forth in the definition of R²; or R¹¹ and R¹², together with the nitrogen to which they are attached, form a 5 to 7 membered heteroalkyl ring that can contain, in addition to the nitrogen atom to which R¹¹ and R¹² are attached, from zero to four heteroatoms selected from the group consisting of nitrogen, sulfur and oxygen, and
the broken lines indicate optional double bonds, with the proviso that when the broken line in G² is a double bond, R⁸ is absent;
(B)
a compound of the formula II 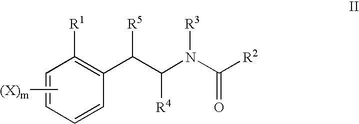
   wherein in Formula II,
R¹ is a group of the formula G¹, G², G³, G⁴, G⁸ or G⁶, wherein G¹, G², G³, G⁴, and G⁶ are each defined as for formula I, and G⁸ is depicted below 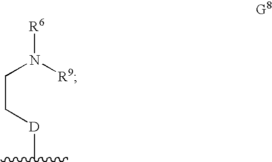
m is 0, 1, 2, 3 or 4;
D is oxygen, sulfur, SO, SO₂, or NR⁷;
a is zero to eight;
p is 1, 2 or 3;
E is oxygen, sulfur, SO or SO₂;
X is hydrogen, chloro, fluoro, bromo, iodo, cyano, (C₁-C₆)alkyl, hydroxy, trifluoromethyl, (C₁-C₆)alkoxy, —S(O)ₜ(C₁-C₆)alkyl wherein t is 0, 1 or 2, —CO₂R¹⁰ or —CONR¹¹R¹²;
R² is —(CH₂)_{y}B, wherein y is 0, 1, 2 or 3, and B is hydrogen, phenyl, naphthyl or a 5 or 6 membered heteroaryl group containing from one to four heteroatoms in the ring, and wherein each of the foregoing phenyl, naphthyl and heteroaryl groups can optionally be substituted with one or more substituents independently selected from chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n is 0, 1 or 2;
R³ and R⁴ are each independently hydrogen, (C₁-C₄)alkyl or —(CH₂)_{q}-J wherein q is 0, 1, 2 or 3, and J is phenyl or naphthyl, wherein said phenyl or naphthyl can be optionally substituted with one to three substituents independently selected from the group consisting of chloro, fluoro, bromo, iodo, (C₁-C₆)alkyl, (C₁-C₆)alkoxy, trifluoromethyl, cyano and —S(O)ₖ(C₁-C₆)alkyl wherein k is 0, 1 or 2;
R⁵ is hydrogen or (C₁-C₃)alkyl;
R⁶ is selected from the group consisting of hydrogen, (C₁-C₆)alkyl optionally substituted with (C₁-C₆)alkoxy or one to three fluorine atoms, or [(C₁-C₄)alkyl]aryl wherein the aryl moiety is phenyl, naphthyl, or heteroaryl-(CH₂)_{q2}—, wherein the heteroaryl moiety is selected from the group consisting of pyridyl, pyrimidyl, benzoxazolyl, benzothiazolyl, benzisoxazolyl and benzisothiazolyl and q2 is zero, one, two, three or four, and wherein said aryl and heteroaryl moieties can optionally be substituted with one or more substituents independently selected from the group consisting of chloro, fluoro, bromo, iodo, (C₁-C₆)alkyl, (C₁-C₆)alkoxy, trifluoromethyl, cyano and —SO_{g}(C₁-C₆)alkyl, wherein g is zero, one or two;
R⁷ is selected from the group consisting of hydrogen, (C₁-C₆)alkyl, [(C₁-C₄)alkyl]aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties can optionally be substituted with one or more substituents independently selected from the group consisting of chloro, fluoro, bromo, iodo, (C₁-C₆)alkyl, (C₁-C₆)alkoxy, trifluoromethyl, —C(═O)—(C₁-C₆)alkyl, cyano and —SOⱼ(C₁-C₆)alkyl, wherein j is zero, one or two;
or R⁶ and R⁷ taken together form a 2 to 4 carbon chain;
R⁸ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that contains, in addition to the nitrogen atom to which R⁶ and R⁹ are attached, from zero to four heteroatoms selected from the group consisting of nitrogen, sulfur and oxygen;
each of R¹⁰, R¹¹ and R¹² is selected, independently, from the groups set forth in the definition of R³; or R¹¹ and R¹², together with the nitrogen to which they are attached, form a 5 to 7 membered heteroalkyl ring that can contain, in addition to the nitrogen atom to which R¹¹ and R¹² are attached, from zero to four heteroatoms selected from the group consisting of nitrogen, sulfur and oxygen, and
each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site;
with the proviso that when B is hydrogen, t is not zero; and
with the proviso that when the broken line in formula G² is a double bond, R⁸ is absent;
and optionally
   (iii) a pharmaceutically acceptable carrier:

Another aspect of the present invention relates to a method of treating one or more CNS disorders in a mammal in need of such treatment by administering thereto the pharmaceutical composition described hereinabove in an amount effective to treat such CNS disorder.

Another aspect of the present invention relates to a kit containing the combination of a compound of Formula I with an atypical antipsychotic, optionally with instructions for use. The compound of Formula I and the atypical antipsychotic compound may either be admixed together in the kit with a pharmaceutical carrier or they may each be in separate compartments within a container. In the latter case, one of the aforementioned components may be admixed together with a pharmaceutical carrier or each may be admixed with a pharmaceutical carrier in separate compartments.

Another aspect of the invention relates to a pharmaceutical composition for treating, for example, a disorder or condition that can be treated by enhancing serotonergic neurotransmission in a mammal, preferably a human, comprising components (i), (ii) and optionally (iii) defined herein;

Another aspect of the invention relates to a method for treating a disorder or condition as defined in the previous paragraphs in a mammal, preferably a human, comprising administering to said mammal in need of such treatment components (i) and (ii) as defined herein.

Another aspect of the invention relates to a method for treating a disorder or condition that can be treated by enhancing serotonergic neurotransmission in a mammal, preferably a human, comprising administering to said mammal in need of such treatment components (i) and (ii) as defined herein.

The pharmaceutical composition of the invention comprises a 5HT_{1B} receptor antagonist of the formula I or II defined herein in combination with an atypical antipyschotic. The compositions and the methods of the invention can be used in an amount to treat CNS disorders.

In the pharmaceutical compositions and methods of the invention, components (i) and (ii) as defined in the previous paragraphs can also be combined with a 5-HT_{1A} antagonist or a pharmaceutically acceptable salt thereof, wherein the amounts of each of components (i), (ii) and the 5-HT_{1A} antagonist or a pharmaceutically acceptable salt thereof are such that the combination of components (i), (ii) and the 5-HT_{1A} antagonist or a pharmaceutically acceptable salt thereof is effective in treating a disorder or condition as defined in the previous paragraphs. For example, the method of the invention can further comprise administering a 5-HT_{1A} antagonist or a pharmaceutically acceptable salt thereof, wherein the amounts of each of components (i), (ii) and the 5-HT_{1A} antagonist or a pharmaceutically acceptable salt thereof are such that the combination of components (i), (ii) and the 5-HT_{1A} antagonist or a pharmaceutically acceptable salt thereof is effective in treating the disorder or condition.

The first component is a compound which acts as an atypical antipsychotic. The atypical antipsychotic, when present in therapeutically effective amounts reduces incidents of EPS. In addition, the atypical antipsychotic can alleviate not only some of the positive symptoms of CNS disorders, such as schizophrenia, but some of the negative symptoms as well, such as emotional unresponsiveness, social withdrawal and the like.

The atypical antipsychotic is a term of art well understood by one of ordinary skill. Typically it exhibits a different and recognizable clinical and pharmacological profile relative to a conventional antipsychotic and exhibit advantages over the conventional antipsychotics. The conventional antipsychotics, such as haloperidol are storage antagonists of dopamine (D2) receptors. The atypical antipsychotics also have D2 antagonist properties, but their binding kinetics to those receptors are different and the antagonist activity to those receptors are relatively weak. However, in addition, they have activity at other receptors, such as 5HT_{2A}, 5HT_{2c} and 5HT_{1d}. The essential feature of an atypical antipsychotic is that it exhibits less acute extrapyramidol symptoms, especially dystonias, associated with therapy as compared to the conventional antipsychotic. For example, atypical antipsychotics have greater efficacy in the treatment of overall psychotherapy in schizophrenics, nonresponsive to typical and antipsychotics; (2) greater efficacy in the treatment of negative symptoms of schizophrenia; (3) less frequent and quantatively smaller increase in serum prolactin concentrations associated with therapy; (4) lower risks of EPS or TD; and (5) improved cognitive functions. See, e.g., Beasley, et al. Neuronsychopharmacology, 14(2): 111, (1996).

Examples of atypical antipsychotics which can be used in the present invention include but are not limited to olanzapine, clozapine, resperidone, sertindole, quetiapine, aripiperazole, amisulpride, asenapine, ziprasidone, mirtazapine and the like.

Ziprasidone, 5-[2[-4-(1,2-benzisothiazol-3-yl)piperazin-1-yl])ethyl]-6-chloro-1,3-dihydro-2H-indol-2-one hydrochloride, is an atypical antipsychotic having in vitro activity as a 5HT1A receptor antagonist, a 5HT2A and dopamine D2 receptor antagonist, and an inhibitor of serotonin and norepinephrine uptake. It is described in U.S. Pat. Nos. 4,831,031, 5,312,295, 6,387,904, 6,245,765, and 6,245,766 and European Patent application EP901781, published Mar. 17, 1999, the contents of all of which is incorporated herein by reference. It is efficacious in the treatment of patients with schizophremia, affective and anxiety symptoms associated with schizoaffective disorder and bipolar disorder.

Olanzapine, which 2-methyl-4-(4-methyl-1-piperazinyl)-10H-thieno [2,3-b][1,5] benzodiazepine, is described in U.S. Pat. No. 5,229,382, the contents of which are incorporated by reference. It is also described as being useful for the treatment of schizophrenia, schizophreniform disorder, acute mania, mild anxiety states and psychosis.

Clozapine, 8-chloro-11-(4-methyl-1-piperazinyl)-5H-dibenzo [b,e][1,4]diazepine is shown to have clinical efficacy in the treatment of schizophrenia. See, Hanes et al., Psychopharmacol Bul. 24, 62 (1998). It is also described in U.S. Pat. No. 3,539,573, the contents of which are incorporated by reference.

Risperidone is 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one. Risperidone and its use in the treatment of psychotic diseases are described in U.S. Pat. No. 4,804,663 which is herein incorporated by reference in its entirety.

Sertindole is 1-[2-[4-[5-chloro-1-(4-fluorophenyl)-1H-indol-3-yl]-1-piperidinyl]ethyl] limidazolidin-2-one. Sertindole is described in U.S. Pat. No. 4,710,500, and its use in the treatment of schizophrenia is described in U.S. Pat. Nos. 5,112,838 and 5,238,945, the contents of all of which are herein incorporated by reference in their entirety.

Quetiapine is 5-[2-(4-dibenzo[b,f][1,4]thiazepin-11-yl-1-piperazinyl)ethyoxy]ethanol. Quetiapine and its activity in assays which demonstrate utility in the treatment of schizophrenia are described in U.S. Pat. No. 4,879,288, which is herein incorporated by reference in its entirety. Quetiapine is typically administered as its (E)-2-butenedioate (2:1) salt.

Aripiprazole, 7-{4-[4-(2,3-dichlorophenyl)-1-piperazinyl-butoxy}-3-, 4-dihydro carbostyril or 7-{4-[4-(2,3-dichlorophenyl)-1 piperazinyl]-butoxy}-3,4-dihydro-2(1 H)-quinolinone, is an atypical antipsychotic agent used for the treatment of schizophrenia and is described in U.S. Pat. No. 4,734,416 and U.S. Pat. No. 5,006,528 both of which are herein incorporated by reference in their entirety.

Amisulpride, which is 4-amino-N-[1-ethyl-2-pyrrolidinyl)methyl]-5-(etylsulfonyl)-2-methoxy benzamide is a known antipsychotic. It exhibits dopamine antagonist activity in rats. See P. Protais, et al. Neuropharmacol, 24, 861 (1985). It is described in U.S. Pat. No. 4,401,822, the contents of which are incorporated by reference.

Asenapine, which is trans-5-chloro-2-methyl-2,3,3a,12b-tetrahydro-1H-dibenz [2,3:6,7]oxepino[4,5-c]pyrrole, is an atypical antipsychotic. Preparation and use of asenapine is described in U.S. Pat. Nos. 4,145,434 and 5,763,476, which are incorporated herein in their entireties by reference.

Mirtazepine, which is 1, 2, 3, 4, 10, 14b-hexa-hydro-2-methyl pyrazino [2,1-a] pyrido [2,3-c]-[2] benzazepine is useful for treatment of major depressive disorders. It is described in U.S. Pat. No. 4,062,848, the contents of which are incorporated by reference.

Asenapine, trans-5-chloro-2-methyl-2,3,3a,12b-tetrahydro-1H-dibenz[2,3:6,7]oxepino[4,5-c]pyrrole. Preparation and use of asenapine is described in U.S. Pat. Nos. 4,145,434 and 5,763,476.

The most preferred atypical antipsychotic is ziprasidone.

Methods of making the second component, the 5-HT_{1B} receptor antagonist described hereinabove, are disclosed U.S. Pat. Nos. 6,462,048; 6,258,953; 6,380,186; and 6,323,229; U.S. Patent Publication Nos. 2002/0091119 and 2003/0083337. These references are incorporated herein by reference.

“Enhancing serotonergic neurotransmission,” as used herein, refers to increasing or improving the neuronal process whereby serotonin is released by a pre-synaptic cell upon excitation and crosses the synapse to stimulate or inhibit the post-synaptic cell.

“Chemical dependency,” as used herein, means an abnormal craving or desire for, or an addiction to a drug. Such drugs are generally administered to the affected individual by any of a variety of means of administration, including oral, parenteral, nasal or by inhalation. Examples of chemical dependencies treatable by the methods of the present invention are dependencies on alcohol, nicotine, cocaine, amphetamine and other psychostimulants, morphine, heroin and other opioid agonists, phenobarbital and other barbiturates, and benzodiazepines such as diazepam and others. “Treating a chemical dependency,” as used herein, means reducing or alleviating such dependency.

A “unit dosage form” as used herein is any form that contains a unit dose of the atypical antipsychotic or a pharmaceutically acceptable salt thereof, of the compound of formula I or formula II or a pharmaceutically acceptable salt thereof, or of the atypical antipsychotic or pharmaceutically acceptable salt thereof and the compound of formula I or formula II or pharmaceutically acceptable salt thereof. A unit dosage form can be, for example, a tablet or a capsule. A unit dose can be an amount which can be predetermined, for example, by a physician.

As used herein, “mammal” means any member of the class Mammalia. As an example, the mammal in need of the treatment can be a human. As another example, the mammal in need of the treatment can be a mammal other than a human.

The methods of this invention also encompass treating the diseases or conditions described herein by the co-administration of two separate pharmaceutical compositions. In this latter embodiment, a first composition comprises an atypical antipsychotic, and a second composition comprises a 5-HT_{1B} receptor antagonist of the formula I or II. These first and second compositions are preferably co-administered either simultaneously, or in a specifically timed manner.

A prodrug of the atypical antipsychotic, of the 5-HT_{1B} receptor antagonist of the formula I or II, or of both the atypical antipsychotic and the 5-HT_{1B} receptor antagonist also can be used in the composition and method of the invention. The term “prodrug” refers to compounds that are drug precursors which, following administration, release the drug in vivo via some chemical or physiological process (e.g., a prodrug on being brought to the physiological pH is converted to the desired drug form). A prodrug of any or all of the atypical antipsychotics or the 5-HT_{1B} receptor antagonists can be used in the methods, kits, and compositions of the instant invention. In general, prodrugs are functional derivatives of these compounds which are readily convertible in vivo. Conventional procedures for the selection and preparation of suitable prodrug derivatives are described, for example, in Design of Prodrugs, ed. H. Bundgaard, Elsevier, 1985 and can be achieved using methods well known to those skilled in the art. All such prodrugs are within the scope of the combinations, pharmaceutical compositions, methods and kits of this invention.

Upon cleavage, exemplary prodrugs release the corresponding free acid (where applicable), and such hydrolyzable ester-forming residues of the prodrugs of this invention include but are not limited to carboxylic acid substituents wherein the free hydrogen is replaced by (C₁-C₄)alkyl, (C₂-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as N,N-dimethylaminoethyl), carbamoyl-(C₁-C₂)alkyl, N,N-di(C₁-C₂)-alkylcarbamoyl-(C₁-C₂)alkyl, piperidino-, pyrrolidino-, or morpholino(C₂-C₃)alkyl, and the like.

The present invention also relates to pharmaceutically acceptable acid addition salts of compounds of the formula I or formula II. The acids which are used to prepare the pharmaceutically acceptable acid addition salts of the aforementioned base compounds of this invention are those which form non-toxic acid addition salts, such as salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [1,1′-methylene-bis-(2-hydroxy-3-naphthoate)]salts.

The invention also relates to base addition salts of formula I or formula II. The chemical bases that can be used as reagents to prepare pharmaceutically acceptable base salts of those compounds of formula I or formula II that are acidic in nature are those that form non-toxic base salts with such compounds. Such non-toxic base salts include, but are not limited to those derived from such pharmacologically acceptable cations such as alkali metal cations, such as potassium and sodium, and alkaline earth metal cations, such as calcium and magnesium, ammonium or water-soluble amine addition salts such as N-methylglucamine-(meglumine), and the lower alkanolammonium and other base salts of pharmaceutically acceptable organic amines.

The compounds of this invention include all stereoisomers, such as cis and trans isomers, and all optical isomers of compounds of the formula I or formula II, such as R and S enantiomers, as well as racemic, diastereomeric and other mixtures of such isomers.

The compounds of this invention can contain C═C double bonds. When such bonds are present, the compounds of the invention exist as cis and trans configurations and as mixtures thereof.

Unless otherwise indicated, the following terms and related variations of same as used herein representatively have the meanings ascribed:

The terms “alkyl” and “alkenyl” referred to herein, as well as the alkyl moieties of other groups referred to herein, such as alkoxy, can be linear or branched, and they can also be cyclic (e.g., cyclopropyl, cyclobutyl, cyclopentyl, or cyclohexyl) or be linear or branched and contain cyclic moieties.

The terms “halo” or “halogen” includes fluorine, chlorine, bromine, and iodine.

The term “a 5 or 6 membered heteroaryl group containing from one to four heteroatoms in the ring”, as used herein, unless otherwise indicated, includes but is not limited to furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indazolyl, isoquinolyl, quinolyl, phthalazinyl, quinoxalinyl, quinazolinyl or benzoxazinyl.

The term “a 5 to 7 membered heteroalkyl ring that can contain from one to four heteroatoms selected from nitrogen, sulfur and oxygen”, as used herein, unless otherwise indicated, includes but is not limited to pyrrolidine, isoxazolidine, 1,3-oxazolidin-3-yl, isothiazolidine, 1,3-thiazolidin-3-yl, 1,2-pyrazolidin-2-yl, 1,3-pyrazolidin-1 -yl, piperidine, thiomorpholine, 1,2-tetrahydrothiazin-2-yl, 1,3-tetrahydrothiazin-3-yl, tetrahydrothiadiazine, morpholine, 1,2-tetrahydrodiazin-2-yl, 1,3-tetrahydrodiazin-1-yl, piperazine.

The following are more specific embodiments of groups G¹ and G² of the compound of formula I: 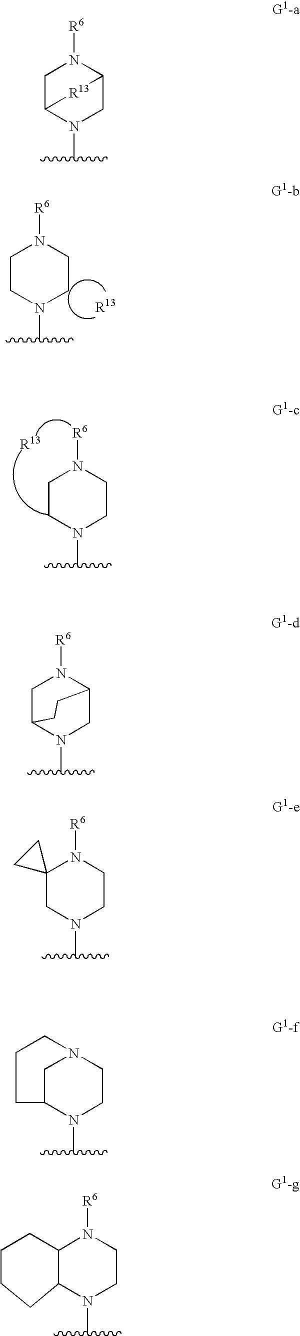 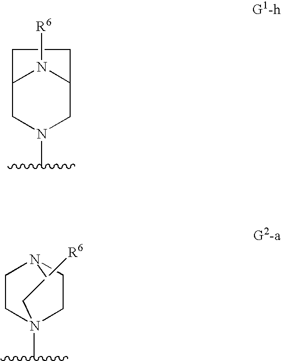
wherein each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site.

Preferred compounds of the formula I include those wherein R¹ is 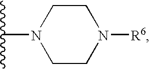
R⁶ is (C₁-C₆)alkyl, such as methyl, and R² is hydrogen.

Other preferred compounds of formula I include those wherein R³ is hydrogen, phenyl or benzyl optionally substituted by chloro, fluoro, bromo, iodo, (C₁-C₆)alkyl or trifluoromethyl.

Other preferred compounds of formula I include those wherein R⁴ is hydrogen or (C₁-C₆)alkyl, such as methyl.

More preferred compounds of formula I include those wherein R¹ is 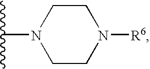
R⁶ is (C₁-C₆)alkyl and R² is hydrogen; R³ is phenyl or benzyl optionally substituted by chloro, fluoro, bromo, iodo, (C₁-C₆)alkyl or trifluoromethyl; and R⁴ is hydrogen or (C₁-C₆)alkyl.

Preferred compounds of the formula I also include those wherein Y, together with the atoms to which it is attached, forms an optionally substituted five to seven membered heterocycle selected from the group consisting of 1,3 thiazolidin-2,4-dion-5-yl, 1,3 imidazolidin-2,4-dion-5-yl, thiomorpholin-3-on-2-yl or morpholin-3-on-2-yl.

Preferred compounds of the formula I also include those wherein R³ is optionally substituted phenyl or —(CH₂)-optionally substituted phenyl, wherein said phenyl groups are optionally substituted with one or more substituents independently selected from the group consisting of chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n in —SOₙ(C₁-C₆)alkyl is zero, one or two.

Preferred compounds of the formula I also include those wherein R⁵ is hydrogen or methyl.

Preferred compounds of the formula I also include those wherein X is hydrogen, fluoro or chloro, preferably wherein X is hydrogen.

Preferred compounds of the formula I also include those wherein R⁴ and R⁵, together with the nitrogen to which they are attached, form a 5 to 7 membered heteroalkyl ring that is selected from the group consisting of pyrrolidine, isoxazolidine, 1,3-oxazolidin-3-yl, isothiazolidine, 1,3-thiazolidin-3-yl, 1,2-pyrazolidin-2-yl, 1,3-pyrazolidin-1-yl, piperidine, thiomorpholine, 1,2-tetrahydrothiazin-2-yl, 1,3-tetrahydrothiazin-3-yl, tetrahydrothiadiazine, morpholine, 1,2-tetrahydrodiazin-2-yl, 1,3-tetrahydrodiazin-1-yl, and piperazine.

Preferred compounds of the formula I also include those wherein m is 0 or 1.

Preferred compounds of the formula II include those wherein R¹ is 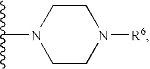
R⁶ is (C₁-C₆)alkyl and R³is hydrogen.

Other preferred compounds of formula II include those wherein R² is phenyl or benzyl optionally substituted by chloro, fluoro, bromo, iodo, (C₁-C₆)alkyl or trifluoromethyl.

Other preferred compounds of formula II include those wherein R⁴ is hydrogen or (C₁-C₆)alkyl.

More preferred compounds of formula II include those wherein R¹ is 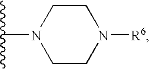
R⁶ is (C₁-C₆)alkyl and R³ is hydrogen; R² is phenyl or benzyl optionally substituted by chloro, fluoro, bromo, iodo, (C₁-C₆)alkyl or trifluoromethyl; and R⁴ is hydrogen or (C₁-C₆)alkyl.

Preferred examples of compounds of component (ii) include:
4-benzyl-2-[2-(4-methylpiperazin-1-yl)-benzylidene]-thiomorpholin-3-one;
4-(3,4-dichlorobenzyl)-2-[2-(4-methylpiperazin-1-yl)-benzylidene]-thiomorpholin-3-one;
2-[2-(4-methylpiperazin-1-yl)-benzylidene]-4-(4-trifluoromethylphenyl)-thiomorpholin-3-one;
2-[2-(4-methylpiperazin-1-yl)-benzylidene]-thiomorpholin-3-one;
4-(3,4-dichlorophenyl)-2-[2-fluoro-6-(4-methylpiperazin-1-yl)-benzylidene]-thiomorpholin-3-one;
4-(3,4-dichlorophenyl)-2-[2-(4-methylpiperazin-1-yl)-benzylidene]-thiomorpholin-3-one;
4-(3,4-dichlorophenyl)-2-[2-(4-methylpiperazin-1-yl)-benzyl]-thiomorpholin-3-one;
4-methyl-2-[2-(4-methylpiperazin-1.-yl)-benzylidene]-thiomorpholin-3-one;
4-(3,4-dichlorophenyl)-2-(2-piperazin-1-ylbenzylidene)-thiomorpholin-3-one;
4-(3,4-dichlorophenyl)-2-[2-(4-methyl-(4-piperazin-1-yl)-benzylidene]-1-oxo thiomorpholin-3-one;
4-(3,4-dichlorophenyl)-2-[2-(4-methyl-4-oxy-piperazin-1-yl)-benzylidene]-thiomorpholin-3-one;
10-[4(3,4-dichlorophenyl)-3-oxo-thiomorpholin-2-yl]-2-methyl-3,42-dihydropyrazino[1,2-a]indol-2-ium;
4-Benzyl-2-[2-(4-methylpiperazin-1-yl)-benzylidene]-1,1-dioxothiomorpholin-3-one;
4-(3,4-Dichlorophenyl)-2-[3-fluoro-2-(4-methylpiperazin-1-yl)-benzylidene]-thiomorpholin-3-one;
4-(3,4-Dichlorophenyl)-2-[5-fluoro-2-(4-methylpiperazin-1-yl)-benzylidene]-thiomorpholin-3-one;
4-(3,4-Dichlorophenyl)-2-[2-(4-methylpiperazin-1-yl)-5-trifluoromethyl-benzylidene]-thiomorpholin-3-one;
4-(3,4-Dichlorophenyl)-2-{2-[4-(2-methoxyethyl)piperazin-1-yl]-benzylidene}thiomorpholin-3-one;
4-(3,4-Dichlorophenyl)-2-[2-(4-isopropylpiperazin-1-yl)-benzylidene]-thiomorpholin-3-one;
4-(3,4-Dichlorophenyl)-2-[2-(4-ethylpiperazin-1-yl)-benzylidene]-thiomorpholin-3-one;
4-(4-Chlorophenyl)-2-[2-(4-methylpiperazin-1-yl)-benzylidene]-thiomorpholin-3-one;
4-(3-Chlorophenyl)-2-[2-(4-methylpiparazin-1-yl)-benzylidene]-thiomorpholin-3-one;
2-[2-Chloro-6-(4-methylpiperazin-1-yl)-benzylidene]-4-(3,4-dichlorophenyl)-thiomorpholin-3-one;
4-(3,4-Dichlorophenyl)-2-[2-(4-methylpiperazin-1-yl)-4-trifluoromethyl-benzylidene]-thiomorpholin-3-one;
4-(3,4-Dichlorophenyl)-2-[2-(4-methylpiperazin-1-yl)-benzylidene]-1-oxo-thiomorphol in-3-one;
4-(3,4-Dichlorophenyl)-2-(5-fluoro-2-piperazin-1-yl-benzylidene)-thiomorpholin-3-one;
4-(3,4-Dichlorophenyl)-2-[3,6-difluoro-2-(4-methylpiperazin-1-yl)-benzylidene]-thiomorpholin-3-one;
4-(3,4-Dichlorophenyl)-2-[2-(3,5-dimethylpiperazin-1-yl)-benzylidene]-thiomorpholin-3-one;
4-Phenyl-2-[2-(3,4,5-trimethylpiperazin-1-yl)-benzylidene]-thiomorpholin-3-one;
2-[5-Fluoro-2-(4-methylpiperazin-1-yl)-benzylidene]-4-phenyl-thiomorpholin-3-one;
4-Benzo[1,3]dioxol-5-yl-2-[2-(3,5-dimethylpiperazin-1-yl)-benzylidene]-thiomorpholin-3-one;
2-[2-(4-tert-Butylpiperazin-1-yl)-benzylidene]-4-(3,4-dichlorophenyl)-thiomorpholin-3-one;
3-[4-(3,4-Dichlorophenyl)-3-oxo-thiomorpholin-2-yl idenemethyl]-6-dimethylamino-2-(4-methylpiperazin-1-yl)-benzonitrile;
4-(3,4-Dichlorophenyl)-2-[2-(3,4,5-trimethylpiperazin-1-yl)-benzylidene]-thiomorpholin-3-one;
4-(3,4-Dichlorophenyl)-2-[5-methyl-2-(4-methylpiperazin-1-yl)-benzylidene]-thiomorpholin-3-one;
2-[4-Chloro-2-(4-methylpiperazin-1-yl)-benzylidene]-4-(3,4-dichlorophenyl)-thiomorpholin-3-one;
4-(3,4-Difluorophenyl)-2-[2-(3,5-dimethylpiperazin-1-yl)-benzylidene]-thiomorpholin-3-one;
4-(2,4-Difluorophenyl)-2-[2-(3,5-dimethylpiperazin-1-yl)-benzylidene]-thiomorpholin-3-one;
2-(4-Bromo-2-(4-muthylpiperazin-1-yl)-benzylidene]-4-(3,4-dichlorophenyl)-thiomorpholin-3-one;
4-(3,4-Dichlorophenyl)-2-[2-(1-methylpyrrolidin-2-ylmethoxy)-benzylidene]-thiomorpholin-3-one;
4-(3,5-Dichlorophenyl)-2-[2-(3,5-dimethylpiperazin-1-yl)-benzylidene]-thiomorpholin-3-one;
4-(3,4-Difluorophenyl)-2-[2-(3,4,5-trimethylpiperazin-1-yl)-benzylidene]-thiomorpholin-3-one;
4-(3,4-Dichlorophenyl)-2-[2-(octahydropyrido[1,2-a]pyrazin-2-yl)-benzylidene]-thiomorpholin-3-one;
2-[2-(4-Cyclopropylpiperazin-1-yl)-benzylidene]-4-pyridin-3-yl-thiomorpholin-3-one;
2-[2-(4-Cyclopropylpiperazin-1-yl)-benzylidene]-4-(3,4-difluorophenyl)-thiomorpholin-3-one;
2-[2-(4-Cyclopropylpiperazin-1-yl)-benzylidene]-4-(3,5-dichlorophenyl)-thiomorpholin-3-one;
4-(3,4-Difluorophenyl)-2-[2-(2,5-dimethylpiperazin-1-yl)-benzylidene]-thiomorpholin-3-one;
4-(3,5-Dichlorophenyl)-2-[2-(2,5-dimethylpiperazin-1-yl)-benzylidene]-thiomorpholin-3-one;
4-(3,4-Dichlorophenyl)-2-[2-(3-methylaminopyrrolidin-1-yl)-benzylidene]-thiomorpholin-3-one;
4-(3,4-Difluorophenyl)-2-[2-(2,4,5-trimethylpiperazin-1-yl)-benzylidene]-thiomorpholin-3-one;
4-Benzo[1,3]dioxol-5-yl-2-[2-(4-cyclopropylpiperazin-1-yl)-benzylidene]-thiomorpholin-3-one;
2-[2-(3,5-Dimethylpiperazin-1-yl)-benzylidene]4-(4-fluorophenyl)-thiomorpholin-3-one;
4-Benzo[1,3]dioxol-5-yl-2-[2-(2,5-dimethylpiperazin-1-yl)-benzylidene]-thiomorpholin-3-one;
2-[2-(3,5-Dimethylpiperazin-1-yl)-benzylidene]-4-phenylthiomorpholin-3-one;
4-(3,4-Dichlorophenyl)-2-[2-(4-methylpiperazin-1-yl)-benzylidene]-thiomorpholin-3-one;
4-(3,4-Dichlorophenyl)-2-[2-(3-dimethylaminopyrrolidin-1-yl)-benzylidene]-thiomorpholin-3-one;
4-(3,4-Dichlorophenyl)-2-[2-(3-dimethylaminopyrrolidin-1-yl)-benzylidene]-thiomorpholin-3-one;
4-(3,4-Dichlorophenyl)-2-[2-(4-methyl-[1,4]diazepan-1-yl)-benzylidene]-thiomorpholin-3-one;
4-(3,4-Dichlorophenyl)-2-[2-(2,4,6-trimethylpiperazin-1-yl)-benzylidene]-thiomorpholin-3-one;
2-[2-(4-Cyclopropylpiperazin-1-yl)-benzylidene]-4-(3,4-dichlorophenyl)-thiomorpholin-3-one;
the (−)-enantiomer of a compound of formula 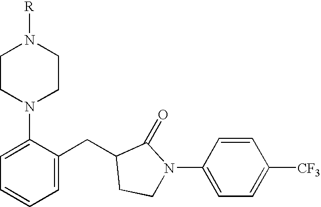
   or a pharmaceutically acceptable salt thereof; wherein R is H or CH₃;
a compound of formula 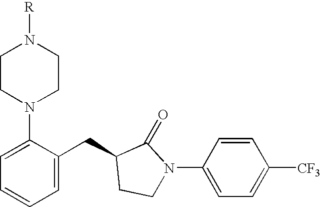
   or a pharmaceutically acceptable salt thereof; wherein R is H or CH₃;
(−)-3(S)-[[2-(4-methyl-1-piperazinyl)phenyl]methyl]-l -[4-(trifluoromethyl)phenyl]-2-pyrrolidinone;
an enantiomeric mixture of (−)-3(S)-[[2-(4-methyl-1-piperazinyl)phenyl]methyl]-1-[4-(trifluoromethyl)phenyl]-2-pyrrolidinone; and (+)-3(R)-[[2-(4-methyl-1-piperazinyl)phenyl]methyl]-1-[4-(trifluoromethyl)phenyl]-2-pyrrolidinone, or pharmaceutically acceptable salts thereof; wherein the ratio of the 3(S)-enantiomer to the (R)-enantiomer is in excess of 2:1, 5:1 or 99:1;
a compound of formula III 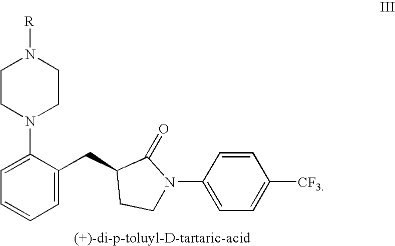
   wherein R is H or CH₃;
3,4-Dichloro-N-(2-[2-(4-methylpiperazin-1-yl)-phenyl]-ethyl)-benzamide;
4-Fluoro-N-(2-[2-(4-methylpiperazin-1-yl)-phenyl]-ethylybenzamide;
N-(2-[2-(4-methylpiperazin-1-yl)-phenyl]-ethyl)-benzamide;
3,4-Dichloro-N-(1-methyl-2-[2-(4-methylpiperazin-1-yl)-phenyl]-ethyl)-benzamide;
3,4-Dichloro-N-(1-methyl-2-[2-(4-methylpiperazin-1-yl)-phenyl]-propyl)-benzamide;
3,4-Dichloro-N-methyl-N-(2-[2-(4-methylpiperazin-1-yl)-phenyl]-ethyl)-benzamide;
N-Benzy-N-(2-[2-(4-methylpiperazin-1-yl)-phenyl]-ethyl)benzamide;
N-(4-chlorobenzyl)-N-(2-[2-(4-methylpiperazin-1-yl)-phenyl]-ethyl)-benzamide;
3,4-Dichloro-N-(2-{2-[methyl-(1-methylpyrolidin-2-ylmethyl)-amino]-phenyl}-ethyl)-benzamide;
3,4-Dichloro-N{2-[2-(1-methyl-octahydro-pyrrolo[2,3-c]pyridin-6-yl)-phenyl]-ethyl}-benzamide;
3,4-Dichloro-N{2-[2-(hexahydro-pyrrolo[1,²-a]pyrazin-2-yliphenyl]-ethyl}-benzamide;
3,4-Dichloro-N{2-[2-(1-methylpiperidin-4-yl)-phenyl]-ethyl)-benzamide;
3,4-Dichloro-N-{2-[2-(2-dimethylaminoethoxy)-phenyl]-ethyl)-benzamide;
3,4-Dichloro-N-{2-[2-(2-dimethylamino-ethylsulfanyl)-phenyl]-ethyl)-benzamide;
3,4-Dichloro-N{2-[2-(2-pyrrolidin-1-ylethoxy)-phenyl]-ethyl}-benzamide;
4-Chloro-N{2-[2-(3-dimethylamino-pyrrolidin-1-yl)-phenyl]-ethyl}-benzamide;
4-Chloro-N-(2-{2-[methyl-(2-morpholin-4-yl-ethyl)-amino]-phenyl)-ethyl)-benzamide;
2-(4-Chloro-phenyl)-N{2-[2-(4-methylpiperazin-1-ylyphenyl]-ethyl)acetamide;
N{2-[2-(4-Methylpiperazin-1-yl)-phenyl]-ethyl}-N-phenylacetamide;
N{2-[2-(4-Methylpiperazin-1-yl)-phenyl]-ethyl}-isonicotinamide;
N{2-[2-(1-Azabicyclo[2.2.2]oct4-yl)phenyl]-ethyl)N-methylbenzamide;
N{2-[2-(1,4-Dimethylpiperidin-4-yl)-phenyl]-ethyl}-4-fluorobenzamide;
4-Fluoro-N-2-[2-(9-methyl-3,9-diazabicyclo[3.3.1]non-3-yl)-phenyl]-ethyl}-benzamide;
N-(2-[2-(1,4-Diazabicyclo[3.3.1]non-4-yl)-phenyl]-ethyl}-N-methylbenzamide;
N-1-Methyl-2-[2-(5-methyl-2,5-diazabicyclo[2.2.1]hept-2-yl)-phenyl]-ethylbenzamide;
2,4-Dichloro-N-methyl-N-1-methyl-2-[2-(3-methyl-3,8-diazabicyclo[3.2.1]oct-8-yl)-phenyl]-ethyl}-benzamide;
N-2-[2-(4-Methyl-octahydroquinoxalin-1-yl)-phenyl]-ethyl}-benzamide;
N{2-[2-(1-Ethylpyrrolidin-2-ylmethoxy)-phenyl]-ethyl}-benzamide;
5-Phenyloxazole-2-carboxylic acid {2-[2-(4-methylpiperazin-1-yl)-phenyl]-ethyl}-amide;
5-Phenylthiophene-2-carboxylic acid {2-[2-(4-methylpiperazin-1-yl)-phenyl]-ethyl}-amide;
5-Methylthiophene-2-carboxylic acid {2-[2-(4-methylpiperazin-1-yl)-phenyl]-ethyl}-amide;
4-Fluoronaphthalene-1-carboxylic acid {2-[2-(4-methylpiperazin-1-yl)-phenyl]-ethyl}-amide;
5-Fluoro-1H-indole-2-carboxylic acid {2-[2-(4-methyl-piperazin-1-ylyphenyl]-ethyl}-amide;
4-Chloro-N-{2-[2-(3,4,5-trimethylpiperazin-1-yl)-phenyl]-ethyl}-benzamide;
3,4-Dichloro-N{2-[2-(2,4,5-trimethylpiperazin-1-yl)-phenyl]-ethyl}-benzamide; and
3,4-Dichloro-N-2-[2-(2,4,6-trimethylpiperazin-1-yl)phenyl]-ethyl}-benzamide.

Methods for making the 5-HT_{1B} receptor antagonists of the formula I or II described above are disclosed in the above-listed patents and published patent applications incorporated by reference herein, including, for example, U.S. Pat. Nos. 6,462,048; 6,258,953; 6,380,186; and 6,323,229; U.S. Patent Publication Nos. 2002/0091119 and 2003/0083337.

In the preferred kits of the present invention, the pharmaceutical composition comprising an atypical antipsychotic is a pharmaceutical composition comprising one of the particularly preferred atypical antipsychotics as defined above, and the pharmaceutical composition comprising a 5-HT_{1B} receptor antagonist is a pharmaceutical composition comprising one of the particularly preferred 5-HT_{1B} receptor antagonists as defined above.

The preferred methods of treatment of the present invention are those methods that employ a particularly preferred atypical antipsychotic and particularly preferred 5-HT_{1B} receptor antagonist as defined above.

Also preferred are those methods that employ a particularly preferred atypical antipsychotic and a particularly preferred 5-HT_{1B} receptor antagonist or a pharmaceutical composition(s) of the present invention, as defined above, for treating CNS disorders.

CNS disorders contemplated for treatment by the present invention include, without limitation, anxiety or psychotic disorders, movement disorders, chemical dependencies, disorders comprising, as a symptom thereof, a deficiency in cognition, or mood disorders or mood episodes.

Non-limiting examples of psychotic disorders include schizophrenia, for example of the paranoid, disorganized, catatonic, undifferentiated, or residual type; schizophreniform disorder; schizoaffective disorder, for example of the delusional type or the depressive type; delusional disorder; substance-induced psychotic disorder, for example psychosis induced by alcohol, amphetamine, cannabis, cocaine, hallucinogens, inhalants, opioids, or phencyclidine; personality disorder of the paranoid type; and personality disorder of the schizoid type.

Non-limiting examples of anxiety disorders include, but are not limited to, panic disorder; agoraphobia; a specific phobia; social phobia; obsessive-compulsive disorder; post-traumatic stress disorder; acute stress disorder; or generalized anxiety disorder.

Non-limiting examples of movement disorders include Huntington's disease and dyskinesia associated with dopamine agonist therapy; Parkinson's disease or restless leg syndrome.

Non-limiting examples of chemical dependencies include alcohol, amphetamine, cocaine, opiate, or nicotine addiction.

Non-limiting examples of disorders comprising, as a symptom thereof, a deficiency in cognition include a subnormal functioning in one or more cognitive aspects such as memory, intellect, or learning and logic ability, in a particular individual relative to other individuals within the same general age population. Also, any reduction in any particular individual's functioning in one or more cognitive aspects, for example as occurs in age-related cognitive decline. Examples of disorders that comprise as a symptom a deficiency in cognition that can be treated according to the present invention are dementia, for example Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; Alzheimer's related dementia; delirium; amnestic disorder; post-traumatic stress disorder; mental retardation; a learning disorder, for example reading disorder, mathematics disorder, post operative cognitive decline, or a disorder of written expression; attention-deficit/hyperactivity disorder; or age-related cognitive decline.

Non-limiting examples of mood disorders or mood episodes include major depressive episode of the mild, moderate or severe type, a manic or mixed mood episode, a hypomanic mood episode; a depressive episode with atypical features; a depressive episode with melancholic features; a depressive episode with catatonic features; a mood episode with postpartum onset; post-stroke depression; major depressive disorder; dysthymic disorder; minor depressive disorder; treatment resistant depression, SSRI-resistant depression, premenstrual dysphoric disorder; post-psychotic depressive disorder of schizophrenia; a major depressive disorder superimposed on a psychotic disorder such as delusional disorder or schizophrenia; a bipolar disorder, for example bipolar I disorder, bipolar II disorder, and cyclothymic disorder. Other CNS disorders involved treatment resistant depression, SSR1 failures, autism and post operative decline.

Other disorders subject to treatment by the invention include those selected from: hypertension, autism, depression (e.g. depression in cancer patients, depression in Parkinson's patients, postmyocardial infarction depression, subsyndromal symptomatic depression, depression in infertile women, pediatric depression, major depression, single episode depression, recurrent depression, child abuse induced depression, and post partum depression), generalized anxiety disorder, phobias (e.g. agoraphobia, social phobia and simple phobias), posttraumatic stress syndrome, avoidant personality disorder, premature ejaculation, eating disorders (e.g. anorexia nervosa and bulimia nervosa), obesity, chemical dependencies (e.g. addictions to alcohol, cocaine, heroin, phenobarbital, nicotine and benzodiazepines), cluster headache, migraine, pain, obsessive-compulsive disorder, panic disorder, memory disorders (e.g. dementia, amnestic disorders, and age-related cognitive decline (ARCD), Parkinson's diseases (e.g. dementia in Parkinson's disease, neuroleptic-induced parkinsonism and tardive dyskinesias), endocrine disorders (e.g. hyperprolactinaemia), vasospasm (particularly in the cerebral vasculature), cerebellar ataxia, gastrointestinal tract disorders (involving changes in motility and secretion), negative symptoms of schizophrenia, schizoaffective disorder, obsessive compulsive disorder, mania, premenstrual syndrome, fibromyalgia syndrome, stress incontinence, Tourelte's syndrome, trichotillomania, kleptomania, male impotence, cancer (e.g. small cell lung carcinoma), chronic paroxysmal hemicrania and headache (associated with vascular disorders).

The present invention also relates to using the pharmaceutical composition of the present invention for treating cognitive function disorders. As used herein this term “Cognitive function” refers to multiple mental process such as learning perception, language, attention, information processing spatial ability and memory (figural and verbal). The term cognitive function disorder refers to a deficit in one or more of the cognitive functions, e.g., memory functions, problem solving, orientation, and/or abstractions that impinges on an individual's ability to function independently. Examples include dementia, cognitive impairment caused by traumatized brain injury, Alzheimer's diseases, age-related memory disorder, vascular dementia, dementia due to other general medical conditions, e.g., Human Immunodeficiency Virus infection, head trauma, Parkinson's disease or Huntington's disease, substance-induced dementia, dementia due to multiple etiologies and the like. See, for example, DSM-IV, 4th ed., pp. 135-180.

The present invention also relates to a method for treating a disorder or condition treatable by modulating serotonergic neurotransmission in a mammal, preferably a human, comprising administering to a mammal in need of such treatment a therapeutically effective amount of a compound of component (i) and component (ii).

Other disorders and conditions subject to treatment by the present invention are delineated in WO 99/52907 to Bright, the disclosure of which is incorporated herein by reference thereto.

The present invention also relates to a pharmaceutical composition for treating the aforesaid disorders/conditions, among others, comprising a therapeutically effective amount of a compound of the invention, including preferably the compound defined by Formula I and the atypical antipsychotic agent and a pharmaceutically acceptable carrier.

Preferably, the combinations of pharmaceutically active compounds of the present invention show a synergistic effect and/or show less side effects, as compared to the individual compounds, when treating a mammal, preferably a human. Thus, in treating a particular disease, at a specific dosage level, the combinations of pharmaceutically active compounds of the present invention show a better activity than the activity which could be expected when administering the individual compounds, less or less severe side effects than could be expected when administering the individual compounds, or a combination of a better activity and of less or less severe side effects than could be expected when administering the individual compounds.

### DETAILED DESCRIPTION OF THE INVENTION

Compounds of the formula I and II or their pharmaceutically acceptable salts, and atypical antipsychotics or their pharmaceutically acceptable salts are hereinafter also referred to, collectively, as “the active compounds.” “The active compounds” are useful in the treatment of Anxiety disorders, schizophrenia, schizophreniform disorder, schizoaffective disorder, delusional disorder, substance-induced psychotic disorder, personality disorder of the paranoid type; personality disorder of the schizoid type, movement disorders involving huntington's disease, dyskinesia associated with dopamine agonist therapy, restless leg syndrome, disorders comprising, as a symptom thereof, a deficiency in cognition, Alzheimer's disease, multi-infarct dementia, alcoholic dementia or other drug-related dementia, dementia associated with intracranial tumors or cerebral trauma, dementia associated with Huntington's disease or Parkinson's disease, AIDS-related dementia, Alzheimer's related dementia, delirium, amnestic disorder, post-traumatic stress disorder, mental retardation, a learning disorder, attention-deficit/hyperactivity disorder, age-related cognitive decline, mood disorders, mood episodes, major depressive episode of the mild, moderate or severe type, a manic or mixed mood episode, a hypomanic mood episode, a depressive episode with atypical features, a depressive episode with melancholic features, a depressive episode with catatonic features, a mood episode with postpartum onset, post-stroke depression, major depressive disorder, dysthymic disorder, minor depressive disorder, treatment resistant depression, SSRI-resistant depression, premenstrual dysphoric disorder, post-psychotic depressive disorder of schizophrenia, a major depressive disorder superimposed on a psychotic disorder, a bipolar disorder, treatment resistant depression, SSR1 failures, autism, operative decline, hypertension, autism, depression, depression in cancer patients, depression in Parkinson's patients, postmyocardial infarction depression, subsyndromal symptomatic depression, depression in infertile women, pediatric depression, major depression, single episode depression, recurrent depression, child abuse induced depression, post partum depression, generalized anxiety disorder, phobias, agoraphobia, social phobia simple phobia, posttraumatic stress syndrome, avoidant personality disorder, premature ejaculation, eating disorders, anorexia nervosa, bulimia nervosa, obesity, chemical dependencies, cluster headache, migraine, pain, obsessive-compulsive disorder, panic disorder, memory disorders, dementia, amnestic disorders, age-related cognitive decline (ARCD), dementia in Parkinson's disease, neuroleptic-induced parkinsonism, tardive dyskinesias, endocrine disorders vasospasm, cerebellar ataxia, gastrointestinal tract disorders, mania, premenstrual syndrome, fibromyalgia syndrome, stress incontinence, Tourette's syndrome, trichotillomania, kleptomania, male impotence, cancer, chronic paroxysmal hemicrania, and headache associated with vascular disorders. Similarly,,the compositions of the present invention are useful in the treatment of the disorders or conditions listed in this paragraph.

The affinities of the compounds of the formula I for the various serotonin-1 receptors can be determined using standard radioligand binding assays as described in the literature. The 5-HT_{1A} affinity can be measured using the procedure of Hoyer et al. (Brain Res., 376, 85 (1986)). The 5-HT_{1B} affinity can be measured using the procedure of Heuring and Peroutka (J. Neurosci., 7, 894 (1987)). The activity of the compounds of the formula I or II at the 5-HT_{1B} binding site, the activity for 5-HT_{1A} binding ability, and the agonist and antagonist activities of the compounds of the formula I or II at 5-HT_{1A} and 5-HT_{1B} receptors can be determined as described in U.S. Pat. No. 6,380,186. All 5-HT_{1B} receptor antagonists that were tested exhibited IC₅₀'s less than 0.60 μM for 5-HT_{1B} affinity and IC₅₀'s less than 1.0 μM for 5-HT_{1A} affinity. Similarly, the activity at the 5-HT_{1B} binding site, the activity for 5-HT_{1A} binding ability, and the agonist and antagonist activities of the compositions of the present invention can be determined using the procedures described for the compounds in formula I in U.S. Pat. No. 6,380,186.

In the present invention, the 5-HT_{1B} receptor antagonists of formula I or II and the atypical antipsychotics can also be further combined with one or more other therapeutic agents, for instance, different antidepressant agents such as tricyclic antidepressants such as amitriptyline, dothiepin, doxepin, trimipramine, butripyline, clomipramine, desipramine, imipramine, iprindole, lofepramine, nortriptyline or protriptyline, monoamine oxidase inhibitors such as isocarboxazid, phenelzine or tranylcyclopramine or monoamine reuptake inhibitors such as fluvoxamine, sertraline, fluoxetine or paroxetine, and/or with antiparkinsonian agents such as dopaminergic antiparkinsonian agents such as levodopa, preferably in combination with a peripheral decarboxylase inhibitor such as benserazide or carbidopa, and/or therapeutic agents which do not appreciably block monoamine uptake or affect monoamine oxidase such as mirtazapine, mianserin, bupropion, lithium salts, antiepileptic drugs such as caramazepine, valproate, lamotrigine, topiramate, gabapentin, pregabalin. It is to be understood that the present invention covers the combination of a 5-HT_{1B} receptor antagonists of formula I or II or a pharmaceutically acceptable salt thereof with an atypical antipsychotic or a pharmaceutically acceptable salt thereof and with one or more such therapeutic agents.

The combination of the compounds of the formula I or II or the pharmaceutically acceptable salts thereof and an atypical antipyschotic, or a pharmaceutically acceptable salt thereof, is also referred herein to as “the active combination.”

Activity of the active combinations as antidepressants and related pharmacological properties can be determined by methods (1)-(3) below, which are described in Koe, B. et al. Journal of Pharmacology and Experimental Therapeutics, 226 (3), 686-700 (1983). Specifically, activity can be determined by studying (1) their ability to affect the efforts of mice to escape from a swim-tank (Porsolt mouse “behavior despair” test), (2) their ability to potentiate 5-hydroxytryptophan-induced behavioral symptoms in mice in vivo, and (3) their ability to block the uptake of serotonin, norepinephrine and/or dopamine by synaptosomal rat brain cells in vitro. The ability of the active combinations to counteract reserpine hypothermia in mice in vivo can be determined according to the methods described in U.S. Pat. No. 4,029,731. The activity of the active combinations as antidepressants and related pharmacological properties also can be determined by methods (4)-(8)) below. Specifically, activity can be determined by studying (4) their ability to reverse the stress-induced decrease in sucrose intake in rodents described in Papp, M. et al., European Journal of Pharmacology, 261, 141-147 (1994), (5) learned helplessness paradigm described in Martin P et al., Life Sciences, 48, 2505-2511 (1991), (6) reversing the behavioral deficits of olfactory bulbectomized rats described in Broekkamp C L et al., Pharmacology, Biochemistry and Behavior, 13, 643-646 (1980), (7) increasing down-regulation or desensitization of beta-adrenergic receptors described in Mishra R. et al., Neuropharmacology, 19, 983-987 (1980), and (8) increasing extracellular levels of serotonin, norepinephrine, and/or dopamine in the prefrontal cortex of freely-moving rodents by in vivo dialysis described in Millan M J et al., European Journal of Neuroscience, 12, 1079-1095 (2000).

Methods that can be used to determine atypical antipsychotic activity of the compounds employed to practice the present invention are as described in U.S. Pat. Nos. 4,831,031 and 4,883,795, both of which can be incorporated herein by reference.

The compositions of the present invention can be formulated in a conventional manner using one or more pharmaceutically acceptable carriers. Thus, the active compounds or the active combinations of the invention can be formulated for oral, buccal, intranasal, parenteral (e.g., intravenous, intramuscular, intraperitoneal, or subcutaneous or through an implant) nasal, vaginal, sublingual, rectal o topical administration or in a form suitable for administration by inhalation or insufflation.

For oral administration, the pharmaceutical compositions can take the form of, for example, tablets or capsules prepared by conventional means with pharmaceutically acceptable excipients such as binding agents such as pregelatinized maize starch, polyvinylpyrrolidone or hydroxypropyl methylcellulose; fillers such as lactose, microcrystalline cellulose or calcium phosphate; lubricants such as magnesium stearate, talc or silica; disintegrants such as potato starch or sodium starch glycolate; or wetting agents such as sodium lauryl sulphate. The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents such as sorbitol syrup, methyl cellulose or hydrogenated edible fats; emulsifying agents such as lecithin or acacia, non-aqueous vehicles such as almond oil, oily esters or ethyl alcohol; and preservatives such as methyl or propyl p-hydroxybenzoates or sorbic acid.

For buccal administration, the composition can take the form of tablets or lozenges formulated in conventional manner.

The active compounds or the active combinations of the invention can be formulated for parenteral administration by injection, including using conventional catheterization techniques or infusion. Formulations for injection can be presented in unit dosage form, for example, in ampoules or in multi-dose containers, with an added preservative. The compositions containing the active combinations can take such forms as suspensions, solutions or emulsions in oily or aqueous vehicles, and can contain formulating agents such as suspending, stabilizing and/or dispersing agents. Alternatively, the active ingredient can be in powder form for reconstitution with a suitable vehicle, for example, sterile pyrogen-free water, before use.

The active compounds or the active combinations of the invention can also be formulated in rectal compositions such as suppositories or retention enemas, for example, containing conventional suppository bases such as cocoa butter or other glycerides. Compositions for vaginal administration are preferably suppositories that can contain, in addition to the active substance, excipients such as cocoa butter or a suppository wax. Compositions for nasal or sublingual administration are also prepared with standard excipients well known in the art.

For intranasal administration or administration by inhalation, the active compounds or the active combinations of the invention are conveniently delivered in the form of a solution or suspension from a pump spray container that is squeezed or pumped by the patient or as an aerosol spray presentation from a pressurized container or a nebulizer, with the use of a suitable propellant, for example, dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. The pressurized container or nebulizer can contain a solution or suspension of the active compounds or the active combinations. Capsules and cartridges, made, for example, from gelatin, for use in an inhaler or insufflator can be formulated containing a powder mix of an active compound and a suitable powder base such as lactose or starch.

The pharmaceutical compositions of the present invention can consist of a combination of immediate release and controlled release characteristics. Such compositions can take the form of combinations of the active ingredients that range in size from nanoparticles to microparticles or in the form of a plurality of pellets with different release rates. The tablet or capsule composition of the present invention can contain a 5-HT_{1B} receptor antagonist of the formula I or II in sustained or controlled release form and the atypical antipsychotic in an immediate release form. Alternatively, the 5-HT_{1B} receptor antagonist can be in immediate release form and the atypical antipsychotic can be in sustained or controlled release form.

An exemplary dose of the active combinations of the invention for oral, parenteral or buccal administration to the average adult human for the treatment of the conditions referred to above, such as depression, ranges from about 0.1 mg to about 200 mg of the active compound of formula I or II and from about 0.1 mg to about 500 mg of the atypical antipsychotic per unit dose which could be administered, for example, 1 to 4 times per day.

The composition of this invention can contain, for example, olanzapine, clozapine, resperidone, sertindole, quetiapine, aripiperazole, amisulpride, asenapine, ziprasidone, mirtazapine as the atypical antipsychotic and 4-benzyl-2-[2-(4-methylpiperazin-1-yl)-benzylidene]-thiomorpholin-3-one, 4-(3,4-dichlorobenzyl)-2-[2-(4-methylpiperazin-1-yl)-benzylidene]-thiomorpholin-3-one, or 2-[2-(4-methylpiperazin-1-yl)-benzylidene]4-(4-trifluoromethyl-phenyl)-thiomorpholin-3-one as the 5-HT_{1B} antagonist. An exemplary daily dose of the atypical antipsychotic in a pharmaceutical composition of this invention for oral, parenteral, rectal or buccal administration to the average adult human for the treatment of the conditions referred to above ranges from about 0.1 mg to about 300 mg of the atypical antipsychotic per unit dose administered 1 to 3 times per day. Exemplary and preferred doses for atypical antipsychotics are determined on a compound by compound basis. 4-benzyl-2-[2-(4-methylpiperazin-1-yl)-benzylidene]-thiomorpholin-3-one, 4-(3,4-dichlorobenzyl)-2-[2-(4-methylpiperazin-1-yl)-benzylidene]-thiomorpholin-3-one, or 2-[2-(4-methylpiperazin-1-yl)-benzylidene]-4-(4-trifluoromethyl-phenyl)-thiomorpholin-3-one can each be present in an amount between about 0.1 mg to about 200 mg, preferably about 0.5 mg to about 10 mg.

Aerosol formulations for treatment of the conditions referred to above, for example, migraine, in the average adult human are preferably arranged so that each metered dose or “puff” of aerosol contains about 20 μg to about 1000 μg of the compound of formula I or II. The overall daily dose with an aerosol will be within the range of about 100 μg to about 10 mg. Administration can be several times daily, for example 2, 3, 4 or 8 times, giving for example, 1, 2 or 3 doses each time. Aerosol formulations containing a compound of formula I or II and an atypical antipsychotic for treatment of the conditions referred to above in the average adult human are preferably arranged so that each metered dose or “puff” of aerosol contains about 100 μg to about 10,000 μg of the compound of formula I formula I or II and about 100 μg to about 30,000 μg of the atypical antiphsychotic. The overall daily dose with an aerosol will be within the range of about 100 μg to about 20,000 mg of the compound of formula I or II and about 100 μg to about 60,000 mg of the atypical antipsychotic. Administration can be several times daily, for example 1, 3, 4 or 8 times, giving for example, 1, 2 or 3 doses each time.

The atypical antipsychotic and the 5-HT_{1B} receptor antagonists of formula I or II can be administered either alone or in combination with pharmaceutically acceptable carriers by either of the routes previously indicated, and such administration can be carried out in both single and multiple dosages. More particularly, this active combination can be administered in a wide variety of different dosage forms, i.e., they can be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of formula I or II are present in such dosage forms at concentration levels ranging from about 0.1% to about 95% by weight of the total composition, i.e., in amounts which are sufficient to provide the desired unit dosage, and an atypical antipsychotic is present in such dosage forms at concentration levels ranging from about 0.1% to about 95% by weight of the total composition, i.e., in amounts which are sufficient to provide the desired unit dosage.

The atypical antipsychotic and the 5-HT_{1B} receptor antagonists of formula I or II can be administered together or separately. When administered separately, the atypical antipsychotics and the compounds of formula I or II can be administered in either order, provided that after administration of the first of the two active ingredients, the second active ingredient is administered within 24 hours or less, preferably 12 hours or less.

A preferred dose ratio of an atypical antipsychotic to a compound of formula I or II in the active combination formulation for oral, parenteral or buccal administration to the average adult human for the treatment of the conditions referred to above is from about 0.001 to about 1000, preferably from about 0.001 to about 100.

It should be understood that the present invention is not limited to the embodiments described herein. Numerous modifications can be made by one skilled in the art having the benefits of the teachings given here. Such modifications should be taken as being encompassed within the scope of the present invention as set forth in the appended claims.

When referring to these preformulation compositions as homogeneous, it is meant that the active ingredients is dispersed evenly throughout the composition so that the composition can be readily subdivided into equally effective unit dosage forms such as tablets, pills and capsules. This solid preformulation composition is then subdivided~into unit dosage forms of the type described above containing from about 0.1 to about 2000 mg of each of the active ingredients of the present invention. Typical unit dosage forms contain from about 1 to about 300 mg, for example about 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The dosage of active ingredients in the compositions and methods of this invention can be varied; however, it is necessary that the amount of the active ingredients in such compositions be such that a suitable dosage form is obtained. The selected dosage depends upon the desired therapeutic effect, on the route of administration, the particular compounds administered, the duration of the treatment, and other factors. All dosage ranges and dosage levels mentioned herein refer to each pharmaceutically active compound present in the pharmaceutical compositions and kits of the present invention, as well as those used in the methods of the present invention. Generally, dosage levels of between about 0.0001 to about 100 mg/kg of body weight daily are administered to humans and other animals, e.g., mammals. A preferred dosage range in humans is about 0.01 to about 5.0 mg/kg of body weight daily which can be administered as a single dose or divided into multiple doses. A preferred dosage range in mammals other than humans is about 0.01 mg/kg to about 10.0 mg/kg of body weight daily which can be administered as a single dose or divided into multiple doses. A more preferred dosage range in mammals other than humans is about 0.1 mg/kg to about 5.0 mg/kg of body weight daily which can be administered as a single dose or divided into multiple doses.

In general, the pharmaceutical compositions, methods and kits of this invention, will be administered at dosages of a therapeutically effective amount of the first and of the second active compound in single or divided doses. The term “therapeutically effective amount” as used herein refers to a sufficient amount of the compound to treat mood disorders and psychotic disorders or conditions at a reasonable benefit/risk ratio applicable to any medical treatment.

The specific therapeutically effective dose level for any particular patient will depend upon a variety of factors including the disorder being treated and the severity of the disorder; activity of the specific compound employed; the specific composition employed; the age. However, some variation in dosage will necessarily occur depending upon the condition of the subject being treated. The person responsible for administration will, in any event, determine the appropriate dose for the individual subject.

The dosage amounts set forth in this description and in the appendant claims can be used, for example, for an average human subject having a weight of about 65 kg to about 70 kg. The skilled practitioner will readily be able to determine any variation in the dosage amount that can be required for a subject whose weight falls outside the 65 kg to 70 kg range, based upon the medical history of the subject. The pharmaceutical combinations can be administered on a regimen of up to 6 times per day, preferably 1 to 3 times per day, such as 2 times per day or once daily.

The present invention also encompasses treatment with a combination of active ingredients which can be administered separately. Accordingly, the invention also relates to combining separate pharmaceutical compositions in kit form. Thus, in one embodiment, the kit comprises two separate pharmaceutical compositions: an atypical antipsychotic or a pharmaceutically acceptable salt of said atypical antipsychotics; and a 5-HT_{1B} receptor antagonist of the formula I or 1I or a pharmaceutically acceptable salt of said 5-HT_{1B} receptor antagonist. The kit also comprises a container for containing the separate compositions such as a divided bottle or a divided foil packet, however, the separate compositions can also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician. An example of such a kit is a so-called blister pack, such as a blister pack that is used in the packaging industry for the packaging of pharmaceutical unit dosage forms, including tablets, capsules, and the like. It can be desirable to provide a memory aid on the kit, e.g., in the form of numbers next to the tablets or capsules whereby the numbers correspond with the days of the regimen which the dosage form so specified should be ingested. Another example of such a memory aid is a calendar printed on the card e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several tablets or capsules to be taken on a given day. Also, a daily dose of an atypical antipsychotic, or a pharmaceutically acceptable salt of said atypical antipsychotics can consist of one tablet or capsule, while a daily dose of the 5-HT_{1B} receptor antagonist of formula I or II or a pharmaceutically acceptable salt of said 5-HT_{1B} receptor antagonist can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

In another embodiment, the present invention comprises kits comprising a pharmaceutical composition, a package, and a package insert. The pharmaceutical composition of these kits contains either atypical antipsychotic or a 5-HT_{1B} receptor antagonist of formula I or II. The kits of the present invention containing a pharmaceutical composition containing an atypical antipsychotic differ from known kits containing a pharmaceutical composition containing an atypical antipsychotic in that on the package and/or on the package insert of the kits it is stated that the pharmaceutical composition is to be administered together with a pharmaceutical composition containing a 5-HT_{1B} receptor antagonist. The kits of the present invention containing a pharmaceutical composition containing a 5-HT_{1B} receptor antagonist of formula I or II differ from known kits containing a pharmaceutical composition containing a 5-HT_{1B} receptor antagonist in that on the package and/or on the package insert of the kits it is stated that the pharmaceutical composition is to be administered together with a pharmaceutical composition containing an atypical antipsychotic.

The term “together with” as used in the immediately preceding paragraph is intended to encompass the simultaneous administration of the two pharmaceutical compositions (e.g., a tablet containing one pharmaceutical composition is to be administered orally while the other pharmaceutical composition is administered by way of infusion, two tablets or capsules are to be swallowed together, etc.). The term “together with” is also intended to include the administration of the two pharmaceutical compositions in a specifically timed manner, i.e., one pharmaceutical composition is to be administered a certain time period after administration of the other pharmaceutical composition. The time period in which the two pharmaceutical compositions are to be administered must be sufficiently short for the atypical antipsychotics and the 5-HT_{1B} receptor antagonist of formula I or II to exhibit their activity contemporaneously, preferably in a synergistic manner. The exact time period depends on the specific compounds of the pharmaceutical compositions, the application route, the kind and severeness of the disease to be treated, the kind, age, and condition of the patient to be treated, etc., and can be determined by a physician using known methods in combination with the disclosure of the present invention. Generally, the two compositions are to be administered within 24 hours or less, such as 12 hours or less, preferably within 5 hours, more preferably within 2 hours, and even more preferably within one hour. Most preferably, the two compositions are to be administered at the same time or one immediately after the other.

The combinations of this invention, i.e., an atypical antipsychotic and a 5-HT_{1B} receptor antagonist, can be tested for conditions such as, for example, migraine, depression, obsessive compulsive disorder, post-traumatic stress disorder (PTSD), and eating disorders according to the procedures described in P. P. A. Humphrey et al., Br. J. Pharmacology, 94, 1128 (1988).

The invention is further illustrated by, but by no means limited to, the following example.

### EXAMPLE 1

A pharmaceutical composition is prepared by combining 4-benzyl-2-[2-(4-methylpiperazin-1-yl)-benzylidene]-thiomorpholin-3-one, 4-(3,4-dichlorobenzyl)-2-[2-(4-methylpiperazin-1-yl)-benzylidene]-thiomorpholin-3-one, or 2-[2-(4-methylpiperazin-1-yl)-benzylidene]-4-(4-trifluoromethyl-phenyl)-thiomorpholin-3-one as the 5-HT_{1B} receptor antagonist with an atypical antipsychotic in a pharmaceutically acceptable carrier. The composition contains about 0.5 mg to about 50 mg of the 5-HT_{1B} receptor antagonist and about 50mg to about 200 mg of the atypical antipsychotic to deliver on a daily basis. The composition is administered to a patient for the treatment of depression on a daily, twice daily, or three times daily basis.

It should be understood that the invention is not limited to the particular embodiments described herein, but that various changes and modifications can be made without departing from the spirit and scope of this novel concept as defined by the following claims.

1. A pharmaceutical composition comprising
(i) an atypical antipsychotic or a pharmaceutically acceptable salt thereof,
(ii) a 5-HT_{1B} receptor antagonist or a pharmaceutically acceptable salt thereof, wherein the 5-HT_{1B} receptor antagonist is selected from the group consisting of
(A) a compound of the formula I— 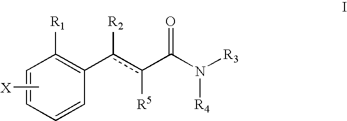
wherein, in formula I:
R¹ is a group of the formula G¹, G², G³, G⁴, G⁵, G⁶ or G⁷ depicted below, 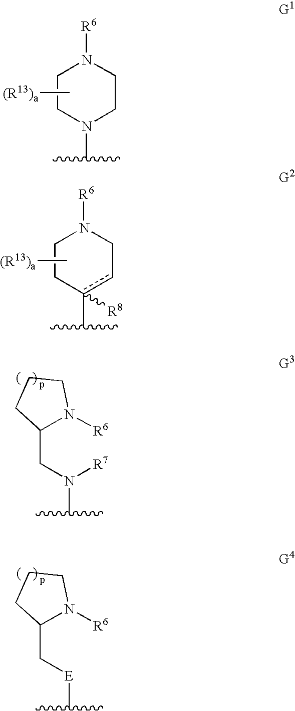
a is zero to eight;
each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site;
E is oxygen, sulfur, SO or SO₂;
X is hydrogen, chloro, fluoro, bromo, iodo, cyano, (C₁-C₆)alkyl, hydroxy trifluoromethyl, (C₁-C₆)alkoxy, —SOₜ(C₁-C₆)alkyl wherein t is zero, one or two, —CO₂R¹⁰ or —CONR¹¹R¹²,
R² is hydrogen, (C₁-C₄)alkyl, phenyl or naphthyl, wherein said phenyl or naphthyl is optionally substituted with one or more substituents independently selected from the group consisting of chloro, fluoro, bromo, iodo, (C₁-C₆)alkyl, (C₁-C₆)alkoxy, trifluoromethyl, cyano and —SOₖ(C₁-C₆)alkyl wherein k is zero, one or two;
R³ is —(CH₂)ₘB, wherein m is zero, one, two or three and B is hydrogen, phenyl, naphthyl or a 5 or 6 membered heteroaryl group containing from one to four hetero-atoms in the ring, and wherein each of the foregoing phenyl, naphthyl and heteroaryl groups is optionally substituted with one or more substituents independently selected from the group consisting of chloro, fluoro, bromo, iodo, (C₁-C₆)alkyl, (C₁-C₆)alkoxy, (C₁-C₆) alkoxy-(C₁-C₆)alkyl-, trifluoromethyl, trifluoromethoxy, cyano, hydroxy, —COOH and —SOₙ(C₁-C₆)alkyl wherein n is zero, one or two;
R⁴ is (C₁-C₆)alkyl or C₆-C₁₀aryl;
or R³ and R⁴ can optionally be taken together with the nitrogen to which they are attached to form a five to seven membered heteroalkyl ring, wherein any two of the carbon atoms of said heteroalkyl ring is optionally replaced with a heteroatom selected from the group consisting of nitrogen, oxygen or sulfur;
R⁵ is hydrogen, (C₁-C₆)alkyl or aryl, wherein aryl is selected from the group consisting of phenyl, naphthyl, pyridyl or pyrimidyl, wherein any of said aryl is optionally independently substituted on any available bonding site by any of the radicals of X;
or R⁵ and R⁴ taken together form a divalent group —Yₙ₂—;
Y is selected from the group consisting of (a) CR⁴R⁵, wherein R⁴ and R⁵ are independently selected from hydrogen, (C₁-C₆)alkyl and trifluoromethyl; (b) a phenylene, naphthylene or a 5 or 6 membered heteroarylene ring comprising containing from one to four hetero-atoms in the heteroarylene ring, and wherein each of the foregoing phenylene, naphthylene and heteroarylene rings can optionally be substituted with one or more substituents independently selected from the group consisting of chloro, fluoro, bromo, iodo, (C₁-C₆)alkyl, (C₁-C₆)alkoxy, (C₁-C₆) alkoxy-(C₁-C₆)alkyl-, trifluoromethyl, trifluoromethoxy, cyano, hydroxy, —COOH and —SOₙ(C₁-C₆)alkyl wherein n is zero, one or two, wherein two adjacent ring atoms of ring Y are also ring atoms of ring A; and (c) an optionally substituted (C₁-C₄) heteroalkyl bridge that, together with the atoms to which it is attached, forms a five to seven membered heterocycle containing two to four heteroatoms selected from the group consisting of 1,3-oxazolidin4-on-5-yl, 1,3-oxazolidin-2,4-dion-5-yl, 4,5-dihydro-1,2-oxazolidin-3-on4-yl, 1,3-thiazolidin-4-on-5-yl, 1,3-thiazolidin-2,4-dion-5-yl, 1,3-pyrazolidin-4-on-5-yl, 1,3-imidazolidin-2,4-dion-5-yl, 1,2-pyrazolidin-3-on-4-yl, 1,2-thiazolidin-1,1,3-trion4-yl, 1,2-thiazolidin-3-on-4-yl, tetrahydro-1,2-oxazin-3-on4-yl, tetrahydro-1,3-oxazin4-on-5-yl, tetrahydro-1,3-oxazin-2,4-dion-5-yl, morpholin-3-on-2-yl, morpholin-3,5-dion-2-yl, 2,3-dihydro-1,4-oxazin-3-on-2-yl, tetrahydro-1,3-thiazin-4-on-5-yl, tetrahydro-1,3-thiazin-2,4-dion-5-yl, tetrahydro-1,2-thiazin-3-on-4-yl, thiomorpholin-3-on-2-yl, thiomorpholin-3,5-dion-2-yl, 2,3-dihydro-1,4-thiazin-3-on-2-yl, hexahydro-1,2-diazin-3-on4-yl, 4,5-dihydro-2H-pyridazin-3-on-4-yl, hexahydro-1,3-diazin-4-on-5-yl, hexahydro-1,3-diazin-2,4-dion-5-yl, piperazin-2-on-3-yl, piperazin-2,6-dion-3-yl, tetrahydro-1,3,4-thiadiazin-5-on-6-yl, 5,6-dihydro-1,3,4-thiadiazin-5-on-6-yl, 1,3,4-oxadiazin-5-on-6-yl, 5,6-dihydro-1,2,4-oxadiazin-5-on-6-yl, tetrahydro-1,2,4-oxadiazin-5-on-6-yl, 1,2,4-triazin-5-on-6-yl, tetrahydro-1,2,4-oxadiazin-5-on-6-yl, 5,6-dihydro-1-2,4-oxadiazin-5-on-6-yl, 1,2,4-oxadiazin-3,5-dion-6-yl, 1,2,4-trazin-6-on-5-yl, hexahydro-1,2-oxazepin-3-on-2-yl, hexahydro-1,3-oxazepin-4-on-5-yl, hexahydro-1,4-oxazepin-3-on-2-yl, hexahydro-1,4-oxazepin-3,5-dion-2-yl, hexahydro-1,4-oxazepin-3,5-dion-6-yl, 2,3,5,6-tetrahydro-1-4-oxazepin-5,7-dion-6-yl, hexahydro-1,4-oxazepin-5-on-6-yl, hexahydro-1,3-oxazepin-2,4-dion-5-yl, hexahydro-1,2-thiazepin-3-on-4-yl, hexahydro-1,4-thiazepin-3-on-2-yl, 2,3,4,5-tetrahydro-1,4-thiazepin-3-on-2-yl, hexahydro-1,4-thiazepin-3,5-dion-2-yl, hexahydro-1,4-thiazepin-3,5-dion-6-yl, 2,3,6,7-tetrahydro-1,4-thiazepin-5-on-6-yl, 6,7-dihydro-1,4-thiazepin-5-on-6-yl, hexahydro-1,3-thiazepin-2,4-dion-5-yl, hexahydro-1,2-diazepin-3-on4-yl, hexahydro-1,3-diazepin-2,4-dion-5-yl, hexahydro-1,4-diazepin-2-on-3-yl, hexahydro-1,4-diazepin-5-on-6-yl, hexahydro-1,4-diazepin-5,7-dion-6-yl, hexahydro-1,3,5-thiadiazepin-3-on-7-yl, 4,5,6,7-tetrahydro-1-3,5-thiadiazepin-6-on-7-yl, and 2,3,5,6-tetrahydro-1,2,4-triazepin-3,5-dion-7-yl; wherein the substituents on any of the carbon atoms capable of supporting an additional bond, of said (C₁-C₄) heteroalkyl bridge, are chloro, fluoro, (C₁-C₆)alkyl, (C₁-C₆)alkoxy, trifluoromethyl or cyano; wherein the substituents on any of the nitrogen atoms capable of supporting an additional bond, of said (C₁-C₄) heteroalkyl bridge, are (C₁-C₆)alkyl or trifluoromethyl,
n2 is one, two, three or four, with the proviso that n2 is one when Y is not CR⁴R⁵;
R⁶ is selected from the group consisting of hydrogen, (C₁-C₆)alkyl optionally substituted with (C₁-C₆)alkoxy or one to three fluorine atoms, or [(C₁-C₄)alkyl]aryl wherein the aryl moiety is phenyl, naphthyl, or heteroaryl-(CH₂)_{q}—, wherein the heteroaryl moiety is selected from the group consisting of pyridyl, pyrimidyl, benzoxazolyl, benzothiazolyl, benzisoxazolyl and benzisothiazolyl and q is zero, one, two, three or four, and wherein said aryl and heteroaryl moieties can optionally be substituted with one or more substituents independently selected from the group consisting of chloro, fluoro, bromo, iodo, (C₁-C₆)alkyl, (C₁-C₆)alkoxy, trifluoromethyl, cyano and —SO_{g}(C₁-C₆)alkyl, wherein g is zero, one or two;
R⁷ is selected from the group consisting of hydrogen, (C₁-C₆)alkyl, [(C₁-C₄)alkyl]aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties can optionally be substituted with one or more substituents independently selected from the group consisting of chloro, fluoro, bromo, iodo, (C₁-C₆)alkyl, (C₁-C₆)alkoxy, trifluoromethyl, —C(═O)—(C₁-C₆)alkyl, cyano and —SOⱼ(C₁-C₆)alkyl, wherein j is zero, one or two;
or R⁶ and R⁷ taken together form a C₂-C₄ alkylene chain;
R⁸ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that contains, in addition to the nitrogen atom to which R⁶ and R⁹ are attached, from zero to four heteroatoms selected from the group consisting of nitrogen, sulfur and oxygen;
and p is one, two, or three;
each of R¹⁰, R¹¹ and R¹² is selected, independently, from the groups set forth in the definition of R²; or R¹¹ and R¹², together with the nitrogen to which they are attached, form a 5 to 7 membered heteroalkyl ring that can contain, in addition to the nitrogen atom to which R¹¹ and R¹² are attached, from zero to four heteroatoms selected from the group consisting of nitrogen, sulfur and oxygen, and
the broken lines indicate optional double bonds, with the proviso that when the broken line in G² is a double bond, R⁸ is absent;
(B)
a compound of the formula II 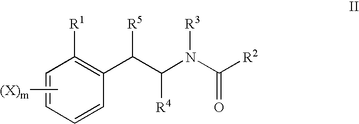
wherein in Formula II,
R¹ is a group of the formula G¹, G² G³ G⁴, G⁸ or G⁶, wherein G¹, G², G³, G⁴ and G⁶ are each defined as for formula I, and G⁸ is depicted below 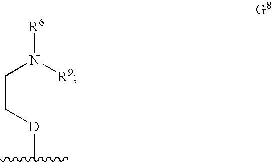
m is 0, 1, 2, 3 or 4;
D is oxygen, sulfur, SO, SO₂, or NR⁷;
a is zero to eight;
p is 1, 2 or 3;
E is oxygen, sulfur, SO or SO₂;
X is hydrogen, chloro, fluoro, bromo, iodo, cyano, (C₁-C₆)alkyl, hydroxy, trifluoromethyl, (C₁-C₆)alkoxy, —S(O)ₜ(C₁-C₆)alkyl wherein t is 0, 1 or 2, —CO₂R¹⁰ or —CONR¹¹R¹²;
R² is —(CH₂)ₜB, wherein t is 0, 1, 2 or 3, and B is hydrogen, phenyl, naphthyl or a 5 or 6 membered heteroaryl group containing from one to four heteroatoms in the ring, and wherein each of the foregoing phenyl, naphthyl and heteroaryl groups can optionally be substituted with one or more substituents independently selected from chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n is 0, 1 or 2;
R³ and R⁴ are each independently hydrogen, (C₁-C₄)alkyl or —(CH₂)_{q}-J wherein q is 0, 1, 2 or 3, and J is phenyl or naphthyl, wherein said phenyl or naphthyl can be optionally substituted with one to three substituents independently selected from the group consisting of chloro, fluoro, bromo, iodo, (C₁-C₆)alkyl, (C₁-C₆)alkoxy, trifluoromethyl, cyano and —S(O)ₖ(C₁-C₆)alkyl wherein k is 0, 1 or 2;
R⁵ is hydrogen or (C₁-C₃)alkyl;
R⁶ is selected from the group consisting of hydrogen, (C₁-C₆)alkyl optionally substituted with (C₁-C₆)alkoxy or one to three fluorine atoms, or [(C₁-C₄)alkyl]aryl wherein the aryl moiety is phenyl, naphthyl, or heteroaryl-(CH₂)_{q2}-, wherein the heteroaryl moiety is selected from the group consisting of pyridyl, pyrimidyl, benzoxazolyl, benzothiazolyl, benzisoxazolyl and benzisothiazolyl and q2 is zero, one, two, three or four, and wherein said aryl and heteroaryl moieties can optionally be substituted with one or more substituents independently selected from the group consisting of chloro, fluoro, bromo, iodo, (C₁-C₆)alkyl, (C₁-C₆)alkoxy, trifluoromethyl, cyano and —SO_{g}(C₁-C₆)alkyl, wherein g is zero, one or two;
R⁷ is selected from the group consisting of hydrogen, (C₁-C₆)alkyl, [(C₁-C₄)alkyl]aryl wherein the aryl moiety is phenyl, naphthyl, or heteroaryl-(CH₂)ᵣ-, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties can optionally be substituted with one or more substituents independently selected from the group consisting of chloro, fluoro, bromo, iodo, (C₁-C₆)alkyl, (C₁-C₆)alkoxy, trifluoromethyl, —C(═O)—(C₁-C₆)alkyl, cyano and —SOⱼ(C₁-C₆)alkyl, wherein j is zero, one or two;
or R⁶ and R⁷ taken together form a 2 to 4 carbon chain;
R⁸ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that contains, in addition to the nitrogen atom to which R⁶ and R⁹ are attached, from zero to four heteroatoms selected from the group consisting of nitrogen, sulfur and oxygen;
each of R¹⁰, R¹¹ and R¹² is selected, independently, from the groups set forth in the definition of R³; or R¹¹ and R¹², together with the nitrogen to which they are attached, form a 5 to 7 membered heteroalkyl ring that can contain, in addition to the nitrogen atom to which R¹¹ and R¹² are attached, from zero to four heteroatoms selected from the group consisting of nitrogen, sulfur and oxygen, and
each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site;
with the proviso that when B is hydrogen, t is not zero; and
with the proviso that when the broken line in formula G² is a double bond, R⁸ is absent;
and optionally
(iii) a pharmaceutically acceptable carrier.

2. The composition of claim 1, wherein in formula I R¹ is 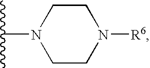
R⁶ is (C₁-C₆)alkyl, such as methyl, and R² is hydrogen.

3. The composition of claim 1, wherein in formula I R³ is hydrogen, phenyl or benzyl optionally substituted by chloro, fluoro, bromo, iodo, (C₁-C₆)alkyl or trifluoromethyl.

4. The composition of claim 1, wherein in formula I R⁴ is hydrogen or (C₁-C₆)alkyl.

5. The composition of claim 4, wherein in formula I R⁴ is methyl.

6. The composition of claim 1, wherein in formula I: R¹ is 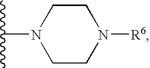
R⁶ is (C₁-C₆)alkyl and R² is hydrogen; R³ is phenyl or benzyl optionally substituted by chloro, fluoro, bromo, iodo, (C₁-C₆)alkyl or trifluoromethyl; and R⁴ is hydrogen or (C₁-C₆)alkyl.

7. The composition of claim 1, wherein in formula I R⁴ and R⁵, together with the nitrogen to which they are attached, form a 5 to 7 membered heteroalkyl ring that is selected from the group consisting of pyrrolidine, isoxazolidine, 1,3-oxazolidin-3-yl, isothiazolidine, 1,3-thiazolidin-3-yl, 1,2-pyrazolidin-2-yl, 1,3-pyrazolidin-1-yl, piperidine, thiomorpholine, 1,2-tetrahydrothiazin-2-yl, 1,3-tetrahydrothiazin-3-yl, tetrahydrothiadiazine, morpholine, 1,2-tetrahydrodiazin-2-yl, 1,3-tetrahydrodiazin-1-yl, and piperazine.

8. The composition of claim 1, wherein in formula I m is 0 or 1.

9. The composition of claim 1, wherein in formula II, R¹ is 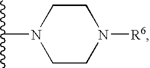
R⁶ is (C₁-C₆)alkyl and R³ is hydrogen.

10. The composition of claim 1, wherein in formula II, R¹ is 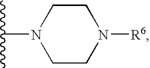
R⁶ is (C₁-C₆)alkyl and R³ is hydrogen; R² is phenyl or benzyl optionally substituted by chloro, fluoro, bromo, iodo, (C₁-C₆)alkyl or trifluoromethyl; and R⁴ is hydrogen or (C₁-C₆)alkyl.

11. The composition of claim 1, wherein the 5-HT_{1B} antagonist is selected from the group consisting of:
4-benzyl-2-[2-(4-methylpiperazin-1-yl)-benzylidene]-thiomorpholin-3-one;
4-(3,4-dichlorobenzyl)-2-[2-(4-methylpiperazin-1-yl)-benzylidene]-thiomorpholin-3-one;
2-[2-(4-methylpiperazin-1-yl)-benzylidene]-4-(4-trifluoromethylphenyl)-thiomorpholin-3-one;
2-[2-(4-methylpiperazin-1-yl)-benzylidene]-thiomorpholin-3-one;
4-(3,4-dichlorophenyl)-2-[2-fluoro-6-(4-methylpiperazin-1-yl)-benzylidene]-thiomorpholin-3-one; and
4-(3,4-dichlorophenyl)-2-[2-(4-methylpiperazin-1-yl)-benzylidene]-thiomorpholin-3-one;
or a pharmaceutically acceptable salt thereof.

12. The composition of claim 1 wherein said atypical antipsychotics is a compound selected from the group consisting of asenapine, olanzapine, clozapine, resperidone, sertindole, quetiapine, aripiperazole, amisulpride, asenapine, ziprasidone, and mirtazapine.

13. A method for treating a disorder or condition selected from the group consisting of Anxiety disorders, schizophrenia, schizophreniform disorder, schizoaffective disorder, delusional disorder, substance-induced psychotic disorder, personality disorder of the paranoid type; personality disorder of the schizoid type, movement disorders involving huntington's disease, dyskinesia associated with dopamine agonist therapy, restless leg syndrome, disorders comprising, as a symptom thereof, a deficiency in cognition, Alzheimer's disease, multi-infarct dementia, alcoholic dementia or other drug-related dementia, dementia associated with intracranial tumors or cerebral trauma, dementia associated with Huntington's disease or Parkinson's disease, AIDS-related dementia, Alzheimer's related dementia, delirium, amnestic disorder, post-traumatic stress disorder, mental retardation, a learning disorder, attention-deficit/hyperactivity disorder, age-related cognitive decline, mood disorders, mood episodes, major depressive episode of the mild, moderate or severe type, a manic or mixed mood episode, a hypomanic mood episode, a depressive episode with atypical features, a depressive episode with melancholic features, a depressive episode with catatonic features, a mood episode with postpartum onset, post-stroke depression, major depressive disorder, dysthymic disorder, minor depressive disorder, treatment resistant depression, SSRI-resistant depression, premenstrual dysphoric disorder, post-psychotic depressive disorder of schizophrenia, a major depressive disorder superimposed on a psychotic disorder, a bipolar disorder, treatment resistant depression, SSR1 failures, autism, operative decline, hypertension, autism, depression, depression in cancer patients, depression in Parkinson's patients, postmyocardial infarction depression, subsyndromal symptomatic depression, depression in infertile women, pediatric depression, major depression, single episode depression, recurrent depression, child abuse induced depression, post partum depression, generalized anxiety disorder, phobias, agoraphobia, social phobia simple phobia, posttraumatic stress syndrome, avoidant personality disorder, premature ejaculation, eating disorders, anorexia nervosa, bulimia nervosa, obesity, chemical dependencies, cluster headache, migraine, pain, obsessive-compulsive disorder, panic disorder, memory disorders, dementia, amnestic disorders, age-related cognitive decline (ARCD), dementia in Parkinson's disease, neuroleptic-induced parkinsonism, tardive dyskinesias, endocrine disorders vasospasm, cerebellar ataxia, gastrointestinal tract disorders, mania, premenstrual syndrome, fibromyalgia syndrome, stress incontinence, Tourette's syndrome, trichotillomania, kleptomania, male impotence, cancer, chronic paroxysmal hemicrania, and headache associated with vascular disorders in a mammal, comprising administering to said mammal in need of such treatment (i) an atypical antipsychotic or a pharmaceutically acceptable salt thereof; and (ii) a 5HT_{1B} receptor antagonist or a pharmaceutically acceptable salt thereof; wherein the amounts of (i) and (ii) administered are together effective in treating said disorder or condition.

14. The method of claim 13 further comprising administering a 5-HT_{1A} antagonist or a pharmaceutically acceptable salt thereof, wherein the amounts of each of components (i), (ii) and the 5-HT_{1A} antagonist or a pharmaceutically acceptable salt thereof are such that the combination of components (i), (ii) and the 5-HT_{1A} antagonist or a pharmaceutically acceptable salt thereof is effective in treating the disorder or condition.

15. The method of claim 13, wherein the disorder or condition is selected from the group consisting of migraine, depression, obsessive compulsive disorder, post-traumatic stress disorder (PTSD), and eating disorders.

16. The method of claim 13, wherein component (ii) is selected from the group consisting of
4-benzyl-2-[2-(4-methylpiperazin-1-yl)-benzylidene]-thiomorpholin-3-one;
4-(3,4-dichlorobenzyl)-2-[2-(4-methylpiperazin-1-yl)-benzylidene]-thiomorpholin-3-one;
2-[2-(4-methylpiperazin-1-yl)-benzylidene]-4-(4-trifluoromethylphenyl)-thiomorpholin-3-one;
2-[2-(4-methylpiperazin-1-yl)-benzylidene]-thiomorpholin-3-one;
4-(3,4-dichlorophenyl)-2-[2-fluoro-6-(4-methylpiperazin-1-yl)-benzylidene]-thiomorpholin-3-one; and
4-(3,4-dichlorophenyl)-2-[2-(4-methylpiperazin-1-yl)-benzylidene]-thiomorpholin-3-one;
or a pharmaceutically acceptable salt thereof.

17. The method of claim 16, wherein component (i) is present in an amount of about 0.1 to about 300 mg and component (ii) is present in an amount of about 0.1 to about 200 mg, wherein component (i) and component (ii) are each administered 1 to 3 times per day.

18. A method for treating a disorder or condition that can be treated by enhancing serotonergic neurotransmission in a mammal, comprising administering to a mammal in need of such treatment (i) an atypical antipsychotic or a pharmaceutically acceptable salt thereof; and (ii) a 5HT_{1B} receptor antagonist or a pharmaceutically acceptable salt thereof; wherein the amounts of (i) and (ii) administered are together effective in treating said disorder or condition.

19. The method of claim 18 wherein component (ii) is used in an amount that is a serotonin receptor antagonizing or agonizing effective amount.

20. The method of claim 18 further comprising administering a 5-HT_{1A} antagonist or a pharmaceutically acceptable salt thereof, wherein the amounts of each of components (i), (ii) and the 5-HT_{1A} antagonist or a pharmaceutically acceptable salt thereof are such that the combination of components (i), (ii) and the 5-HT_{1A} antagonist or a pharmaceutically acceptable salt thereof is effective in treating the disorder or condition.

21. The method of claim 18, wherein the disorder or condition is selected from the group consisting of migraine, depression, obsessive compulsive disorder, post-traumatic stress disorder (PTSD), and eating disorders.

22. The method of claim 18, wherein the component (i) is present in an amount of about 0.1 to about 300 mg and component (ii) is present in an amount of about 0.1 to about 200 mg, wherein component (i) and component (ii) are each administered 1 to 3 times per day.

23. A pharmaceutical composition comprising (i) an atypical antipsychotic or a pharmaceutically acceptable salt thereof; and (ii) a 5HT_{1B} antagonist or pharmaceutically acceptable salt thereof; wherein the amounts of (i) and (ii) in said composition are together therapeutically effective; and wherein said composition further comprises a pharmaceutically acceptable carrier.
